SEQUENCE LISTING (41 sequences)

SEQ ID NO: 1 - DNA, 20 bp, Artificial Sequence
  Antisense Oligonucleotide
ggcggcatgtctattttgta

SEQ ID NO: 2 - DNA, 20 bp, Artificial Sequence
  Antisense Oligonucleotide
cggcatgtctattttgtaaa

SEQ ID NO: 3 - DNA, 20 bp, Artificial Sequence
  Antisense Oligonucleotide
gcggcatgtctattttgtaa

SEQ ID NO: 4 - DNA, 5952 bp, Homo sapiens
gatgttatctgctggcagcagaaggttcgctccgagcggagctccagaagctcctgacaagagaaagacagattgagatagagatagaaagagaaagagagaaagagacagcagagcgagagcgcaagtgaaagaggcaggggagggggatggagaatattagcctgacggtctagggagtcatccaggaacaaactgaggggctgcccggctgcagacaggaggagacagagaggatctattttagggtggcaagtgcctacctaccctaagcgagcaattccacgttggggagaagccagcagaggttgggaaagggtgggagtccaagggagcccctgcgcaaccccctcaggaataaaactccccagccagggtgtcgcaagggctgccgttgtgatccgcagggggtgaacgcaaccgcgacggctgatcgtctgtggctgggttggcgtttggagcaagagaaggaggagcaggagaaggagggagctggaggctggaagcgtttgcaagcggcggcggcagcaacgtggagtaaccaagcgggtcagcgcgcgcccgccagggtgtaggccacggagcgcagctcccagagcaggatccgcgccgcctcagcagcctctgcggcccctgcggcacccgaccgagtaccgagcgccctgcgaagcgcaccctcctccccgcggtgcgctgggctcgcccccagcgcgcgcacacgcacacacacacacacacacacacacgcacgcacacacgtgtgcgcttctctgctccggagctgctgctgctcctgctctcagcgccgcagtggaaggcaggaccgaaccgctccttctttaaatatataaatttcagcccaggtcagcctcggcggcccccctcaccgcgctcccggcgcccctcccgtcagttcgccagctgccagccccgggaccttttcatctcttcccttttggccggaggagccgagttcagatccgccactccgcacccgagactgacacactgaactccacttcctcctcttaaatttatttctacttaatagccactcgtctctttttttccccatctcattgctccaagaatttttttcttcttactcgccaaagtcagggttccctctgcccgtcccgtattaatatttccacttttggaactactggccttttctttttaaaggaattcaagcaggatacgtttttctgttgggcattgactagattgtttgcaaaagtttcgcatcaaaaacaacaacaacaaaaaaccaaacaactctccttgatctatactttgagaattgttgatttcttttttttattctgacttttaaaaacaacttttttttccacttttttaaaaaatgcactactgtgtgctgagcgcttttctgatcctgcatctggtcacggtcgcgctcagcctgtctacctgcagcacactcgatatggaccagttcatgcgcaagaggatcgaggcgatccgcgggcagatcctgagcaagctgaagctcaccagtcccccagaagactatcctgagcccgaggaagtccccccggaggtgatttccatctacaacagcaccagggacttgctccaggagaaggcgagccggagggcggccgcctgcgagcgcgagaggagcgacgaagagtactacgccaaggaggtttacaaaatagacatgccgcccttcttcccctccgaaactgtctgcccagttgttacaacaccctctggctcagtgggcagcttgtgctccagacagtcccaggtgctctgtgggtaccttgatgccatcccgcccactttctacagaccctacttcagaattgttcgatttgacgtctcagcaatggagaagaatgcttccaatttggtgaaagcagagttcagagtctttcgtttgcagaacccaaaagccagagtgcctgaacaacggattgagctatatcagattctcaagtccaaagatttaacatctccaacccagcgctacatcgacagcaaagttgtgaaaacaagagcagaaggcgaatggctctccttcgatgtaactgatgctgttcatgaatggcttcaccataaagacaggaacctgggatttaaaataagcttacactgtccctgctgcacttttgtaccatctaataattacatcatcccaaataaaagtgaagaactagaagcaagatttgcaggtattgatggcacctccacatataccagtggtgatcagaaaactataaagtccactaggaaaaaaaacagtgggaagaccccacatctcctgctaatgttattgccctcctacagacttgagtcacaacagaccaaccggcggaagaagcgtgctttggatgcggcctattgctttagaaatgtgcaggataattgctgcctacgtccactttacattgatttcaagagggatctagggtggaaatggatacacgaacccaaagggtacaatgccaacttctgtgctggagcatgcccgtatttatggagttcagacactcagcacagcagggtcctgagcttatataataccataaatccagaagcatctgcttctccttgctgcgtgtcccaagatttagaacctctaaccattctctactacattggcaaaacacccaagattgaacagctttctaatatgattgtaaagtcttgcaaatgcagctaaaattcttggaaaagtggcaagaccaaaatgacaatgatgatgataatgatgatgacgacgacaacgatgatgcttgtaacaagaaaacataagagagccttggttcatcagtgttaaaaaatttttgaaaaggcggtactagttcagacactttggaagtttgtgttctgtttgttaaaactggcatctgacacaaaaaaagttgaaggccttattctacatttcacctactttgtaagtgagagagacaagaagcaaattttttttaaagaaaaaaataaacactggaagaatttattagtgttaattatgtgaacaacgacaacaacaacaacaacaacaaacaggaaaatcccattaagtggagttgctgtacgtaccgttcctatcccgcgcctcacttgatttttctgtattgctatgcaataggcacccttcccattcttactcttagagttaacagtgagttatttattgtgtgttactatataatgaacgtttcattgcccttggaaaataaaacaggtgtataaagtggagaccaaatactttgccagaaactcatggatggcttaaggaacttgaactcaaacgagccagaaaaaaagaggtcatattaatgggatgaaaacccaagtgagttattatatgaccgagaaagtctgcattaagataaagaccctgaaaacacatgttatgtatcagctgcctaaggaagcttcttgtaaggtccaaaaactaaaaagactgttaataaaagaaactttcagtcagaataagtctgtaagtttttttttttctttttaattgtaaatggttctttgtcagtttagtaaaccagtgaaatgttgaaatgttttgacatgtactggtcaaacttcagaccttaaaatattgctgtatagctatgctataggttttttcctttgttttggtatatgtaaccatacctatattattaaaatagatggatatagaagccagcataattgaaaacacatctgcagatctcttttgcaaactattaaatcaaaacattaactactttatgtgtaatgtgtaaatttttaccatattttttatattctgtaataatgtcaactatgatttagattgacttaaatttgggctctttttaatgatcactcacaaatgtatgtttcttttagctggccagtacttttgagtaaagcccctatagtttgacttgcactacaaatgcattttttttttaataacatttgccctacttgtgctttgtgtttctttcattattatgacataagctacctgggtccacttgtcttttcttttttttgtttcacagaaaagatgggttcgagttcagtggtcttcatcttccaagcatcattactaaccaagtcagacgttaacaaatttttatgttaggaaaaggaggaatgttatagatacatagaaaattgaagtaaaatgttttcattttagcaaggatttagggttctaactaaaactcagaatctttattgagttaagaaaagtttctctaccttggtttaatcaatatttttgtaaaatcctattgttattacaaagaggacacttcataggaaacatctttttctttagtcaggtttttaatattcagggggaaattgaaagatatatattttagtcgatttttcaaaaggggaaaaaagtccaggtcagcataagtcattttgtgtatttcactgaagttataaggtttttataaatgttctttgaaggggaaaaggcacaagccaatttttcctatgatcaaaaaattctttctttcctctgagtgagagttatctatatctgaggctaaagtttaccttgctttaataaataatttgccacatcattgcagaagaggtatcctcatgctggggttaatagaatatgtcagtttatcacttgtcgcttatttagctttaaaataaaaattaataggcaaagcaatggaatatttgcagtttcacctaaagagcagcataaggaggcgggaatccaaagtgaagttgtttgatatggtctacttcttttttggaatttcctgaccattaattaaagaattggatttgcaagtttgaaaactggaaaagcaagagatgggatgccataatagtaaacagcccttgtgttggatgtaacccaatcccagatttgagtgtgtgttgattatttttttgtcttccacttttctattatgtgtaaatcacttttatttctgcagacattttcctctcagataggatgacattttgttttgtattattttgtctttcctcatgaatgcactgataatattttaaatgctctattttaagatctcttgaatctgttttttttttttttaatttgggggttctgtaaggtctttatttcccataagtaaatattgccatgggaggggggtggaggtggcaaggaaggggtgaagtgctagtatgcaagtgggcagcaattatttttgtgttaatcagcagtacaatttgatcgttggcatggttaaaaaatggaatataagattagctgttttgtattttgatgaccaattacgctgtattttaacacgatgtatgtctgtttttgtggtgctctagtggtaaataaattatttcgatgatatgtggatgtctttttcctatcagtaccatcatcgagtctagaaaacacctgtgatgcaataagactatctcaagctggaaaagtcataccacctttccgattgccctctgtgctttctcccttaaggacagtcacttcagaagtcatgctttaaagcacaagagtcaggccatatccatcaaggatagaagaaatccctgtgccgtctttttattcccttatttattgctatttggtaattgtttgagatttagtttccatccagcttgactgccgaccagaaaaaatgcagagagatgtttgcaccatgctttggctttctggttctatgttctgccaacgccagggccaaaagaactggtctagacagtatcccctgtagccccataacttggatagttgctgagccagccagatataacaagagccacgtgctttctggggttggttgtttgggatcagctacttgcctgtcagtttcactggtaccactgcaccacaaacaaaaaaacccaccctatttcctccaatttttttggctgctacctacaagaccagactcctcaaacgagttgccaatctcttaataaataggattaataaaaaaagtaattgtgactcaa

SEQ ID NO: 5 - DNA, 20 bp, Artificial Sequence
  antisense oligonucleotide
ggcggcatgtctattttgta

SEQ ID NO: 6 - DNA, 20 bp, Artificial Sequence
  antisense oligonucleotide
ggcggcatgtctattttgta

SEQ ID NO: 7 - DNA, 20 bp, Artificial Sequence
  antisense oligonucleotide
ggcggcatgtctattttgta

SEQ ID NO: 8 - DNA, 20 bp, Artificial Sequence
  antisense oligonucleotide
ggcggcatgtctattttgta

SEQ ID NO: 9 - DNA, 20 bp, Artificial Sequence
  antisense oligonucleotide
ggcggcatgtctattttgta

SEQ ID NO: 10 - DNA, 20 bp, Artificial Sequence
  antisense oligonucleotide
ggcggcatgtctattttgta

SEQ ID NO: 11 - DNA, 20 bp, Artificial Sequence
  antisense oligonucleotide
ggcggcatgtctattttgta

SEQ ID NO: 12 - DNA, 20 bp, Artificial Sequence
  antisense oligonucleotide
ggcggcatgtctattttgta

SEQ ID NO: 13 - DNA, 20 bp, Artificial Sequence
  antisense oligonucleotide
ggcggcatgtctattttgta

SEQ ID NO: 14 - DNA, 20 bp, Artificial Sequence
  antisense oligonucleotide
ggcggcatgtctattttgta

SEQ ID NO: 15 - DNA, 20 bp, Artificial Sequence
  antisense oligonucleotide
ggcggcatgtctattttgta

SEQ ID NO: 16 - DNA, 20 bp, Artificial Sequence
  antisense oligonucleotide
cggcatgtctattttgtaaa

SEQ ID NO: 17 - DNA, 20 bp, Artificial Sequence
  antisense oligonucleotide
cggcatgtctattttgtaaa

SEQ ID NO: 18 - DNA, 20 bp, Artificial Sequence
  antisense oligonucleotide
cggcatgtctattttgtaaa

SEQ ID NO: 19 - DNA, 20 bp, Artificial Sequence
  antisense oligonucleotide
cggcatgtctattttgtaaa

SEQ ID NO: 20 - DNA, 20 bp, Artificial Sequence
  antisense oligonucleotide
cggcatgtctattttgtaaa

SEQ ID NO: 21 - DNA, 20 bp, Artificial Sequence
  antisense oligonucleotide
cggcatgtctattttgtaaa

SEQ ID NO: 22 - DNA, 20 bp, Artificial Sequence
  antisense oligonucleotide
cggcatgtctattttgtaaa

SEQ ID NO: 23 - DNA, 20 bp, Artificial Sequence
  antisense oligonucleotide
cggcatgtctattttgtaaa

SEQ ID NO: 24 - DNA, 20 bp, Artificial Sequence
  antisense oligonucleotide
cggcatgtctattttgtaaa

SEQ ID NO: 25 - DNA, 20 bp, Artificial Sequence
  antisense oligonucleotide
cggcatgtctattttgtaaa

SEQ ID NO: 26 - DNA, 20 bp, Artificial Sequence
  antisense oligonucleotide
cggcatgtctattttgtaaa

SEQ ID NO: 27 - DNA, 20 bp, Artificial Sequence
  antisense oligonucleotide
gcggcatgtctattttgtaa

SEQ ID NO: 28 - DNA, 20 bp, Artificial Sequence
  antisense oligonucleotide
gcggcatgtctattttgtaa

SEQ ID NO: 29 - DNA, 20 bp, Artificial Sequence
  antisense oligonucleotide
gcggcatgtctattttgtaa

SEQ ID NO: 30 - DNA, 20 bp, Artificial Sequence
  antisense oligonucleotide
gcggcatgtctattttgtaa

SEQ ID NO: 31 - DNA, 20 bp, Artificial Sequence
  antisense oligonucleotide
gcggcatgtctattttgtaa

SEQ ID NO: 32 - DNA, 20 bp, Artificial Sequence
  antisense oligonucleotide
gcggcatgtctattttgtaa

SEQ ID NO: 33 - DNA, 20 bp, Artificial Sequence
  antisense oligonucleotide
gcggcatgtctattttgtaa

SEQ ID NO: 34 - DNA, 20 bp, Artificial Sequence
  antisense oligonucleotide
gcggcatgtctattttgtaa

SEQ ID NO: 35 - DNA, 20 bp, Artificial Sequence
  antisense oligonucleotide
gcggcatgtctattttgtaa

SEQ ID NO: 36 - DNA, 20 bp, Artificial Sequence
  antisense oligonucleotide
gcggcatgtctattttgtaa

SEQ ID NO: 37 - DNA, 20 bp, Artificial Sequence
  antisense oligonucleotide
gcggcatgtctattttgtaa

SEQ ID NO: 38 - DNA, 18 bp, Artificial Sequence
  Primer
tgtgcagaaggatggagt

SEQ ID NO: 39 - DNA, 20 bp, Artificial Sequence
  Primer
ctggtgcttctctcaggata

SEQ ID NO: 40 - DNA, 21 bp, Artificial Sequence
  Primer
cagcacactcgatatggacca

SEQ ID NO: 41 - DNA, 20 bp, Artificial Sequence
  Primer
cctcgggctcaggatagtct

(19) United States
(12) Patent Application Publication
(10) Pub. No.: US 2025/0340882 A1
(43) Pub. Date: Nov. 6, 2025

(54) ANTISENSE OLIGONUCLEOTIDES

(71) Applicant: AUTOTELIC BIO INC., Cheongju-si, Chungcheongbuk-do (KR)

(72) Inventors: Ji Hye KOO, Suwon-si, Gyeonggi-do (KR); Hye Jeong KWON, Seongnam-si, Gyeonggi-do (KR); Ji-Hye KIM, Gimpo-si, Gyeonggi-do (KR); Jun Eui PARK, Seongnam-si, Gyeonggi-do (KR); Soo Jeong HONG, Chungju-si, Chungcheongbuk-do (KR); Chang Hee PARK, Hwaseong-si, Gyeonggi-do (KR); Tae Hun KIM, Sejong (KR)

(73) Assignee: AUTOTELIC BIO INC., Cheongju-si, Chungcheongbuk-do (KR)

(21) Appl. No.: 18/706,036

(22) PCT Filed: Mar. 22, 2022
(86) PCT No.: PCT/KR2022/003884

§ 371 Date: Apr. 30, 2024

(30) Foreign Application Priority Data
Nov. 1, 2021 (KR) 10-2021-0148357

(51) Int. Cl. C12N 15/113; A61P 35/00

(52) U.S. Cl. CPC C12N 15/1136; A61P 35/00; C12N 2310/11; C12N 2310/14; C12N 2310/315; C12N 2310/321

(57) ABSTRACT

The present invention relates to novel antisense oligonucleotides including at least one modified nucleotide from among oligonucleotides of SEQ ID NOs: 1 to 3. The novel oligonucleotides exhibit an excellent effect of inhibiting the expression of TGF-β2 protein, an excellent effect of killing cancer cells, and improved stability in plasma. Therefore, the oligonucleotides of the present invention can be effectively used as a pharmaceutical composition for treating diseases related to TGF-β2 expression.

### TECHNICAL FIELD

The present disclosure relates to antisense oligonucleotides and a pharmaceutical composition including the same.

### BACKGROUND ART

After information in DNA sequences of cells is transcribed into mRNA, proteins are produced through a process in which tRNA transfers corresponding amino acids based on information of mRNA from ribosomes followed by ligation thereof via peptide bonds.

If there is a single strands of DNA or RNA having a sequence complementary to the mRNA in the course of this gene expression, this complementary DNA or RNA can bind to the mRNA to form a double chain, thereby blocking protein production.

An oligonucleotide of the complementary DNA or RNA that can bind to mRNA (sense RNA), which can be transcribed and translated into a protein as described above, forming a double chain to inhibit its function is called an antisense RNA or antisense oligonucleotide.

### DISCLOSURE OF THE INVENTION

### Technical Goals

As a result of research to develop an antisense oligonucleotide with improved ability of inhibiting TGF-β mRNA expression, the inventors of the present invention found that a novel antisense oligonucleotide in which structure of some sugars was modified among antisense oligonucleotides exhibited excellent TGF-β inhibitory effects and high stability.

Accordingly, the present disclosure is intended to provide a novel antisense oligonucleotide and a pharmaceutical composition containing the same.

### Technical Solutions

The present disclosure relates to a novel antisense oligonucleotide including at least one modified nucleoside from among an oligonucleotide of 5′-GGCGG CATGT CTATT TTGTA-3′ represented by SEQ ID NO: 1; an oligonucleotide of 5′-CGGCA TGTCT ATTTT GTAAA-3′ represented by SEQ ID NO: 2; or an oligonucleotide of 5′-GCGGC ATGTC TATTT TGTAA-3′ represented by SEQ ID NO: 3.

Specifically, the present disclosure relates to an antisense oligonucleotide of any one of SEQ ID NOS: 1 to 3, wherein the antisense oligonucleotide has a nucleotide in which at least one nucleoside of the oligonucleotide is modified.

Herein, the nucleotide with the modified nucleoside may be a 2′-O-methoxyethyl (MOE)-modified nucleotide, a 2′-fluoro (F)-modified nucleotide, a 2′-O-methyl (0-Me)-modified nucleotide, a locked nucleic acid (LNA)-modified nucleotide, an ethylene-bridged nucleic acid (ENA)-modified nucleotide, an (R/S)-constrained ethyl (cET)-modified nucleotide, or a polyalkylene oxide (e.g., triethylene glycol (TEG))-modified nucleotide. (Hereinafter, unless otherwise specified, nucleotides with nucleosides modified as described above are referred to as “modified nucleotides”)

For example, the antisense oligonucleotide according to the present disclosure may have a 2′ position on a ribose pentose of a modified nucleoside in a 2′-O-methoxyethyl (MOE) form, a 2′-fluoro form, or a 2′-O-methyl form, or may be in the form of LNA or ENA in which oxygen at the 2′ position on the ribose pentose of the modified nucleoside is connected to carbon at a 4′ position or in the form of cET where a bridge that connects oxygen at the 2′ position and carbon at the 4′ position of the LNA is substituted with a methyl group.

A structure of the modified nucleoside illustrated above may be represented as follows:

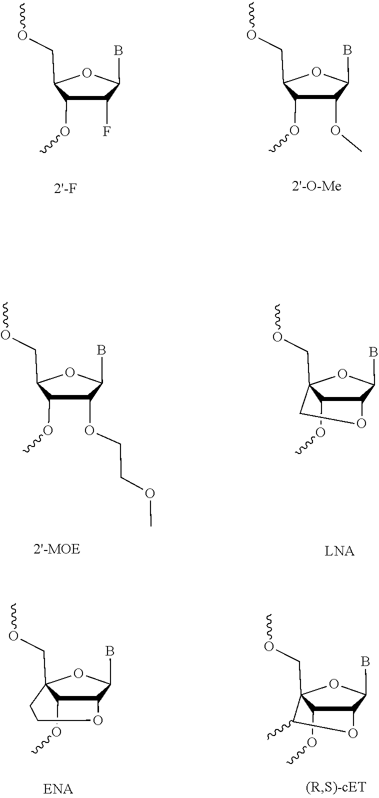

The oligonucleotide of the present disclosure includes one or more modified nucleosides at a 5′-end and/or a 3′ end. For example, the oligonucleotide includes one or more modified nucleosides at the 5′-end or 3′-end of the oligonucleotide of the present disclosure. Thus, a nucleotide having at least one modified nucleoside at the 5′-end or 3′-end of the oligonucleotide of the present disclosure, i.e., a “modified nucleotide” may be included.

Specifically, the oligonucleotide according to the present disclosure is an oligonucleotide in which one or more nucleotides at the 5′-end or one or more nucleotides at the 3′-end of the oligonucleotide are modified nucleotides.

In addition, the oligonucleotide according to the present disclosure is an oligonucleotide in which one or more nucleotides at the 5′-end and one or more nucleotides at the 3′-end of the oligonucleotide are modified nucleotides.

In the present disclosure, the first to n^{th} nucleotides at the 5′-end of the oligonucleotide or the first to m^{th} nucleotides at the 3′-end of the oligonucleotide may be the modified nucleotides.

In addition, in the present disclosure, the first to n^{th} nucleotides at the 5′-end of the oligonucleotide and the first to m^{th} nucleotides at the 3′-end of the oligonucleotide may be the modified nucleotides.

Herein, n and m are each independently an integer of 1 to 10, preferably n and m are each independently an integer of 1 to 9, and more preferably n and m are each independently an integer of 1 to 7.

In addition, the oligonucleotide of the present disclosure may further include a modified nucleotide between the modified nucleosides present at the 5′-end or the 3′-end.

In other words, the oligonucleotide may further include one or more modified nucleotides between the (n+1)^{th} nucleotide at the 5′-end and the (m+1)^{th} nucleotide at the 3′-end of the oligonucleotide.

A preferred example of the oligonucleotide of the present disclosure may be any one of antisense oligonucleotides indicated by 1 to 30 in the following Table.

In Table 1 below, underlined, bold nucleotides indicate modified nucleotides.

TABLE 1

| | | NM001135599 | | | | Sequence |
|---|---|---|---|---|---|---|
| No | Substance | Start | End | Modification | Motif | (5′→3′) |
| | A002 | 1695 | 1676 | | | G*G*C*G*G*C*A*T*G*T*C*T*A*T*T*T*T*G*T*A |
| 1 | A002-01M | 1695 | 1676 | MOE | 4-12-4 | G*G*C*G*G*C*A*T*G*T*C*T*A*T*T*T*T*G*T*A |
| 2 | A002-02M | 1695 | 1676 | MOE | 3-14-3 | G*G*C*G*G*C*A*T*G*T*C*T*A*T*T*T*T*G*T*A |
| 3 | A002-03M | 1695 | 1676 | MOE | 4-14-2 | G*G*C*G*G*C*A*T*G*T*C*T*A*T*T*T*T*G*T*A |
| 4 | A002-04M | 1695 | 1676 | MOE | 5-10-5 | G*G*C*G*G*C*A*T*G*T*C*T*A*T*T*T*T*G*T*A |
| 5 | A002-05M | 1695 | 1676 | MOE | 5-11-4 | G*G*C*G*G*C*A*T*G*T*C*T*A*T*T*T*T*G*T*A |
| 6 | A002-06M | 1695 | 1676 | MOE | 5-12-3 | G*G*C*G*G*C*A*T*G*T*C*T*A*T*T*T*T*G*T*A |
| 7 | A002-07M | 1695 | 1676 | MOE | 5-13-2 | G*G*C*G*G*C*A*T*G*T*C*T*A*T*T*T*T*G*T*A |
| 8 | A002-08M | 1695 | 1676 | MOE | 4-11-5 | G*G*C*G*G*C*A*T*G*T*C*T*A*T*T*T*T*G*T*A |
| 9 | A002-09M | 1695 | 1676 | MOE | 3-12-5 | G*G*C*G*G*C*A*T*G*T*C*T*A*T*T*T*T*G*T*A |
| 10 | A002-10M | 1695 | 1676 | MOE | 2-13-5 | G*G*C*G*G*C*A*T*G*T*C*T*A*T*T*T*T*G*T*A |
| | A003 | 1693 | 1674 | | | C*G*G*C*A*T*G*T*C*T*A*T*T*T*T*G*T*A*A*A |
| 11 | A003-01M | 1693 | 1674 | MOE | 4-12-4 | C*G*G*C*A*T*G*T*C*T*A*T*T*T*T*G*T*A*A*A |
| 12 | A003-02M | 1693 | 1674 | MOE | 3-14-3 | C*G*G*C*A*T*G*T*C*T*A*T*T*T*T*G*T*A*A*A |
| 13 | A003-03M | 1693 | 1674 | MOE | 4-14-2 | C*G*G*C*A*T*G*T*C*T*A*T*T*T*T*G*T*A*A*A |
| 14 | A003-04M | 1693 | 1674 | MOE | 5-10-5 | C*G*G*C*A*T*G*T*C*T*A*T*T*T*T*G*T*A*A*A |
| 15 | A003-05M | 1693 | 1674 | MOE | 5-11-4 | C*G*G*C*A*T*G*T*C*T*A*T*T*T*T*G*T*A*A*A |
| 16 | A003-06M | 1693 | 1674 | MOE | 5-12-3 | C*G*G*C*A*T*G*T*C*T*A*T*T*T*T*G*T*A*A*A |
| 17 | A003-07M | 1693 | 1674 | MOE | 5-13-2 | C*G*G*C*A*T*G*T*C*T*A*T*T*T*T*G*T*A*A*A |
| 18 | A003-08M | 1693 | 1674 | MOB | 4-11-5 | C*G*G*C*A*T*G*T*C*T*A*T*T*T*T*G*T*A*A*A |
| 19 | A003-09M | 1693 | 1674 | MOE | 3-12-5 | C*G*G*C*A*T*G*T*C*T*A*T*T*T*T*G*T*A*A*A |
| 20 | A003-10M | 1693 | 1674 | MOE | 2-13-5 | C*G*G*C*A*T*G*T*C*T*A*T*T*T*T*G*T*A*A*A |
| | A004 | 1694 | 1675 | | | G*C*G*G*C*A*T*G*T*C*T*A*T*T*T*T*G*T*A*A |
| 21 | A004-01M | 1694 | 1675 | MOE | 4-12-4 | G*C*G*G*C*A*T*G*T*C*T*A*T*T*T*T*G*T*A*A |
| 22 | A004-02M | 1694 | 1675 | MOE | 3-14-3 | G*C*G*G*C*A*T*G*T*C*T*A*T*T*T*T*G*T*A*A |
| 23 | A004-03M | 1694 | 1675 | MOE | 4-14-2 | G*C*G*G*C*A*T*G*T*C*T*A*T*T*T*T*G*T*A*A |
| 24 | A004-04M | 1694 | 1675 | MOE | 5-10-5 | G*C*G*G*C*A*T*G*T*C*T*A*T*T*T*T*G*T*A*A |
| 25 | A004-05M | 1694: | 1675 | MOE | 5-11-4 | G*C*G*G*C*A*T*G*T*C*T*A*T*T*T*T*G*T*A*A |
| 26 | A004-06M | 1694 | 1675 | MOE | 5-12-3 | G*C*G*G*C*A*T*G*T*C*T*A*T*T*T*T*G*T*A*A |
| 27 | A004-07M | 1694 | 1675 | MOE | 5-13-2 | G*C*G*G*C*A*T*G*T*C*T*A*T*T*T*T*G*T*A*A |
| 28 | A004-08M | 1694 | 1675 | MOE | 4-11-5 | G*C*G*G*C*A*T*G*T*C*T*A*T*T*T*T*G*T*A*A |
| 29 | A004-09M | 1694 | 1675 | MOE | 3-12-5 | G*C*G*G*C*A*T*G*T*C*T*A*T*T*T*T*G*T*A*A |
| 30 | A004-10M | 1694 | 1675 | MOE | 2-13-5 | G*C*G*G*C*A*T*G*T*C*T*A*T*T*T*T*G*T*A*A |

The modified nucleotide of the oligonucleotide in Table 1 (indicated in underlined, bold letters) is a 2′-O-methoxyethyl (MOE)-modified nucleotide.

Here, Genflank Access No. NM_001135599 in Table 1 is known as the sequence that expresses TGF-β2, as shown below (see SEQ ID NO: 4):

| Genebank NM001135599 | |
|---|---|
| 1 | gatcttatct gctggcagtca gaaggttcgc tccgagcgga gctccagaag ctcctgacaa |
| 61 | gagaaagaca gattgagata gagatagaaa gagaaagaga gaaagagaca gtagascgag |
| 121 | agcgcaagtg aaagcggcag gggagggggc tcgagaatat tagcctgacg gtctagggcg |
| 181 | tcatccagga acaaactgag gggctgcccg gctgcagaca ggaggagaca gagcggatct |
| 241 | attttagcgt ggcaagtgcc tacctaccct aagcgagcaa ttccaccttc gcgagaagcc |
| 301 | agcagaggtt gggaaagggt gggagtccaa gggagcccct gcccaacccc ctcaggaata |
| 361 | aaactcccca gccagggtgt cgcaagggct gccgttgtga tccgcagggg gtgaacgcas |
| 421 | ccgcgacggc tgatcgtctg tcgctgggtt ggcgtttgga gcaagagaag gaggagcagg |
| 481 | agaaggaggg agctggagcc tggaagcgtt tgcaagcggc ggcggcagca acgtggagta |
| 541 | accaagcggg tcagcgcgcg cccgccaggg tgtaggccac ggagcgcagc tcccagagca |
| 601 | ggatccgcgc cgcctcagca gcctctgcgg cccctgcggc acccgaccga gtaccgagcg |
| 661 | ccctgcgaag cgcaccctcc tccccccggt gcgctgggct cgcccccagc gcgcgcacac |
| 721 | gcacacacac acacacaccc acacacgcac gcacacacgt gtgcgcttct ctgctccgga |
| 781 | gctgctgctg ctcctgctct cagcgccgca gtggaaggca ggaccgaacc gctccttctt |
| 841 | taaatatata aatttcagcc caggtcagcc tcggcggccc ccctcaccgc gctcccggcg |
| 901 | cccctcccgt cagttcgcca gctgccagcc ccgggacctt ttcatctctt cccttttggc |
| 961 | cggaggagcc gagttcagat ccgccactcc gcacccgaga ctgacacact gaactccact |
| 1021 | tcctcctctt aaatttattt ctacttaata gccactcgtc tctttttttc cccatctcat |
| 1081 | tgctccaaga atttttttct tcttactcgc caaagtcagg gttccctctg cccgtcccgt |
| 1141 | attaatattt ccacttttgg aactactggc cttttctttt taaaggaatt caagcaggat |
| 1201 | acgtttttct gttgggcatt gactagattg tttgcaaaag tttcgcatca aaaacaacaa |
| 1261 | caacaaaaaa ccaaacaact ctccttgatc tatactttga gaattgttga tttctttttt |
| 1321 | ttattctgac ttttaaaaac aacttttttt tccacttttt taaaaaatgc actactgtgt |
| 1381 | gctgagcgct tttctgatcc tgcatctggt cacggtcgcg ctaagcctgt ctacctgcag |
| 1441 | cacactcgat ctggcccagt tcatgcgcaa gaggatcgcg gcgatccgcg ggcagatcct |
| 1501 | gagcaagctg aagctcacca gtcccccaga agactatcct gagcccgagg aagtcccccc |
| 1561 | ggaggtgatt tccatctaca acagcaccag ggacttgctc caggagacgg cgagccggag |
| 1621 | ggcggccgcc tgcgagcgcg agaggagcga cgaagagtac tacgccaagg aggtttacaa |
| 1681 | aatagacatg ccgcccttct tcccctccga aactgtctgc ccagttgtta caacaccctc |
| 1741 | tggctcagtg ggcagcttgt gctccagaca gtcccaggtg ctctgtgggt accttgatgc |
| 1801 | catcccgccc actttctaca gaccctactt cagaattgtt cgatttgacg tctcagcaat |
| 1861 | ggagaagaat gcttccaatt tggtgaaagc agagttcaga gtctttcgtt tgcagaaccc |
| 1921 | aaaagccaga gtgcctgaac aacggattga gctatatcag attctcaagt ccaaagattt |
| 1981 | aacatctcca acccagcgct acatcgacag caaagttgtg aaaacaagag cagaaggcga |
| 2041 | atggctctcc ttcgatgtaa ctgatgctgt tcatgaatgg cttcaccata aagacaggaa |
| 2101 | cctgggattt aaaataagct tacactgtcc ctgctgcact tttgtaccat ctaataatta |
| 2161 | catcatccca aataaaagtg aagaactaga agcaagattt gcaggtattg atggcacctc |
| 2221 | cacatatacc agtggtgatc agaaaactat aaagtccact aggaaaaaaa acagtgggaa |
| 2281 | gaccccacat ctcctgctaa tgttattgcc ctcctacaga cttgagtcac aacagaccaa |
| 2341 | ccggcggaag aagcgtgctt tggatgcggc ctattgcttt agaaatgtgc aggatcattg |
| 2401 | ctgcctacgt ccactttaca ttgatttcaa gagggatcta gggtggaaat ggatacacga |
| 2461 | acccaaaggg tacaatgcca acttctgtgc tggagcatgc ccgtatttat ggagttcaga |
| 2521 | cactcagcac agcagggtcc tgagcttata taataccata aatccagaag catctgcttc |
| 2581 | tccttgctgc gtgtcccaag atttagaacc tctaaccatt ctctactaca ttggcaaaac |
| 2641 | accccagatt gaacagcttt ctaatatgat tgtaaagtct tgcaaatgca gctaaaattc |
| 2701 | ttggaaaagt ggcaagacca aaatgacaat gatgatgata atgatgatga cgacgacaac |
| 2761 | gatgatgctt gtaacaagaa aacataagag agccttggtt catcagtgtt aaaaaatttt |
| 2821 | tgaaaaggcg gtactagttc agacactttg gaagtttgtg ttctgtttgt taaaactggc |
| 2821 | tgaaaaggcg gtactagttc agacactttg gaagtttgtg ttctgtttgt taaaactggc |
| 2881 | atctgacaca aaaaaagttg aaggccttat tctacatttc acctactttg taagtgagag |
| 2941 | agacaagaag caaatttttt ttaaagaaaa aaataaacac tggaagaatt tattagtgtt |
| 3001 | aattatgtga acaacgacaa caacaacaac aacaacaaac aggaaaatcc cattaagtgg |
| 3061 | agttgctgta cgtaccgttc ctatcccgcg cctcacttga tttttctgta ttgctatgca |
| 3121 | ataggcaccc ttcccattct tactcttaga gttaacagtg agttatttat tgtgtgttac |
| 3181 | tatataatga acgtttcatt gcccttggaa aataaaacag gtgtataaag tggagaccca |
| 3241 | atactttgcc agaaactcat ggatggctta aggaacttga actcaaacga gccagaaaaa |
| 3301 | aagaggtcat attaatggga tgaaaaccca agtgagttat tatatgaccg agaaagtctg |
| 3361 | cattaagata aagaccctga aaacacatgt tatgtatcag ctgcctaagg aagcttcttg |
| 3421 | taaggtccaa aaactaaaaa gactgttaat aaaagaaact ttcagtcaga ataagtctgt |
| 3481 | aagttttttt ttttcttttt aattgtaaat ggttctttgt cagtttagta aaccagtgaa |
| 3541 | atgttgaaat gttttgacat gtactggtca aacttcagac cttaaaatat tgctgtatag |
| 3601 | ctatgctata ggttttttcc tttgttttgg tatatgtaac catacctata ttattaaaat |
| 3661 | agatggatat agaagccagc ataattgaaa acacatctgc agatctcttt tgcaaactat |
| 3721 | tcaatcaaaa cattaactac tttatgtgta atgtgtaaat ttttaccata ttttttatat |
| 3781 | tctgtaataa tgtcaactat gatttagatt gacttaaatt tgggctcttt ttaatgatca |
| 3841 | ctcacaaatg tatgtttctt ttagctggcc agtacttttg agtaaagccc ctatagtttg |
| 3901 | acttgcacta caaatgcatt ttttttttaa taacatttgc cctacttgtg ccttctgttt |
| 3961 | ctttcattat tatgacataa gctacctggg tccacttgtc ttttcttttt tttgtttcac |
| 4021 | agaaaagatg ggttcgagtt cagtggtctt catcttccaa gcatcattac taaccaagtc |
| 4081 | agacgttaac aaatttttat gttaggaaaa ggaggaatgt tatagataca tagaaaattg |
| 4141 | aagtaaaatg ttttcatttt agcaaggatt tagggttcta actaaaactc agaatcttta |
| 4201 | ttgagttaag aaaagtttct ctaccttggt ttaatcaata tttttgtaaa atcctattgt |
| 4261 | tattacaaag aggacacttc ataggaaaca tctttttctt tagtcaggtt tttaatattc |
| 4321 | agggggaaat tgaaagatat atattttagt cgatttttca aaaggggaaa aaagtccagg |
| 4381 | tcagcataag tcattttgtg tatttcactg aagttataag gtttttataa atgttctttg |
| 4441 | aaggggaaaa ggcacaagcc aatttttcct atgatcaaaa aattctttct ttcctctgag |
| 4501 | tgagagttat ctatatctga ggctaaagtt taccttgctt taataaataa tttgccacat |
| 4561 | cattgcagaa gaggtatcct catgctgggg ttaatagaat atgtcagttt atcacttgtc |
| 4621 | gcttatttag ctttaaaata aaaattaata ggcaaagcaa tggaatattt gcagtttcac |
| 4681 | ctaaagagca gcataaggag gcgggaatcc aaagtgaagt tgtttgatat ggtctacttc |
| 4741 | ttttttggaa tttcctgacc attaattaaa gaattggatt tgcaagtttg aaaactggaa |
| 4801 | aagcaagaga tgggatgcca taatagtaaa cagcccttgt gttggatgta acccaatccc |
| 4861 | agatttgagt gtgtgttgat tatttttttg tcttccactt ttctattatg tgtaaatcac |
| 4921 | ttttatttct gcagacattt tcctctcaga taggatgaca ttttgttttg tattattttg |
| 4981 | tctttcctca tgaatgcact gataatattt taaatgctct attttaagat ctcttgaatc |
| 5041 | tgtttttttt ttttttaatt tgggggttct gtaaggtctt tatttcccat aagtaaatat |
| 5101 | tgccatggga ggggggtgga ggtggcaagg aaggggtgaa gtgctagtat gcaagtgggc |
| 5161 | agcaattatt tttgtgttaa tcagcagtac aatttgatcg ttggcatggt taaaaaatgg |
| 5221 | aatataagat tagctgtttt gtattttgat gaccaattac gctgtatttt aacacgatgt |
| 5281 | atgtctgttt ttgtggtgct ctagtggtaa ataaattatt tcgatgatat gtggatgtct |
| 5341 | ttttcctacc agtaccatca tcgagtctag aaaacacctg tgatgcaata agactatctc |
| 5401 | aagctggaaa agtcatacca cctttccgat tgccctctgt gctttctccc ttaaggacag |
| 5461 | tcacttcaga agtcatgctt taaagcacaa gagtcaggcc atatccatca aggatagaag |
| 5521 | aaatccctgt gccgtctttt tattccctta tttattgcta tttggtaatt gtttgagatt |
| 5581 | tagtttccat ccagcttgac tgccgaccag aaaaaatgca gagagatgtt tgcaccatgc |
| 5641 | tttggatttc tggttctatg ttctgccaac gccagggcca aaagaactgg tctagacagt |
| 5701 | atcccctgta gccccataac ttggatagtt gctgagccag ccagatataa caagagccac |
| 5761 | gtgctttctg gggttggttg tttgggatca gctacttgcc tgtcagtttc actggtacca |
| 5821 | ctgcaccaca aacaaaaaaa cccaccctat ttcctccaat ttttttggct gctacctaca |
| 5881 | agaccagact cctcaaacga gttgccaatc tcttaataaa taggcttaat aaaaaaagta |
| 5941 | attgtgactc aa |

For example, an A002 oligonucleotide in Table 1 represents an antisense oligonucleotide that binds complementarily to SEQ ID NO. 1695 to 1676 in the NM_001135599 sequence, and an A002-01M oligonucleotide represents a nucleotide in which 4 nucleotides at 5′ position and 4 nucleotides at 3′ position among 20 nucleotides in A002 oligonucleotides are modified (preferably, a 2′-O-methoxyethyl (MOE)-modified nucleotide).

In the oligonucleotide of the present disclosure, the bond between nucleosides, regardless of whether the nucleoside is modified, may be either phosphodiester or phosphothioate.

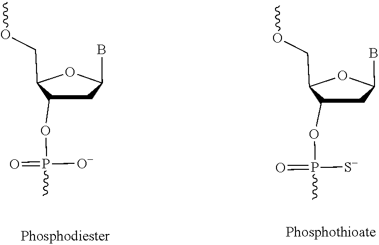

The * mark between nucleotides in the oligonucleotides in Table 1 above indicates phosphothioate.

The oligonucleotides of the present disclosure may form salts with cations. Therefore, “oligonucleotide” as used herein should be understood as a concept that also includes pharmaceutically acceptable salts of oligonucleotides.

Modification of nucleosides as described above in the present disclosure may be prepared via organochemical synthesis methods known in the art to which the present disclosure pertains.

The present disclosure also relates to a pharmaceutical composition including the antisense oligonucleotide modified as described above.

For example, the present disclosure relates to a pharmaceutical composition which includes the antisense oligonucleotide modified as described above and is to prevent or treat diseases (e.g., malignant tumors, benign tumors, immune diseases, fibrosis, or ophthalmic diseases) related to overexpression of TGF-β2 proteins.

### Advantageous Effects

When a solid tumor cell line was treated with an oligonucleotide of the present disclosure, it was found that more superior effects of inhibiting expression of TGF-β2 proteins and outstanding cancer cell killing effects were derived, and a stability in plasma was improved as well.

Therefore, the oligonucleotide of the present disclosure may be useful as a pharmaceutical composition for treating diseases related to expression of TGF-β, such as malignant tumors, benign tumors, immune diseases, fibrosis, and ophthalmic diseases.

### BRIEF DESCRIPTION OF DRAWINGS

FIG. 1 is a graph showing changes in expression of TGF-β2 mRNA by treating A549 with 20 nM ASO of the present disclosure.

FIGS. 2 and 3 are graphs showing changes in expression of TGF-β2 mRNA by treating A549 and PANC-1 with 1 μM ASO of the present disclosure.

FIGS. 4 to 6 shows uptake degrees of ASO of the present disclosure presented in graphs for each of A549, PANC-1, and A2058 cell lines.

FIG. 7 shows a result of an experiment to determine whether ASO of the present disclosure has a stability improvement effect.

FIG. 8 is a graph showing a TGF-β2 mRNA level and a protein expression level after treatment of ASO of the present disclosure by concentration for an A549 cell line.

FIGS. 9 to 11 show TGF-β2 mRNA levels upon treatment of ASO of the present disclosure by concentration for each of A549, PANC-1, and A2058 cell lines.

FIGS. 12 to 18 show TGF-β2 mRNA levels upon treatment of ASO of the present disclosure for cell lines from various carcinomas.

FIGS. 19 to 21 show tumor growth curves upon administration of ASO of the present disclosure into xenograft mice transplanted with an A2058 cell line.

### BEST MODE FOR CARRYING OUT THE INVENTION

When a solid tumor cell line was treated with an oligonucleotide of the present disclosure, it was found that more superior effects of inhibiting expression of TGF-β2 mRNA and excellent anticancer effects to suppress growth of cancer cells were derived, and a stability in plasma was improved as well.

Hereinafter, the present disclosure will be described in detail based on Examples. However, the following Examples are merely for illustrating the present disclosure, and the spirit or scope of the present disclosure is not limited thereby.

### 1. Experimental Methods (Common)

a. Cell Culture

As shown in Table 2 below, according to cell types, media such as RPMI 1640 (10% FBS, 1% antibiotic-antimycotic), DMEM (10% FBS, 1% antibiotic-antimycotic), or McCoy's 5A (10% FBS, 1% antibiotic-antimycotic) were used, and cells were cultured at 37° C. in the presence of 5.0% CO₂.

TABLE 2

| Tissue | Cell line | Diagnosis name | Composition of culture media |
|---|---|---|---|
| Lung | A549 | Carcinoma | RPMI1640 + 10% FBS + 1% Antibiotics |
| Pancreas | Panc-1 | Epithelioid | DMEM + 10% FBS + 1% Antibiotics |
| | | Carcinoma | |
| Pancreas | Capan-2 | Adenocarcinoma | RPMI1640 + 10% FBS + 1% Antibiotics |
| Pancreas | MIA PaCa-2 | Carcinoma | DMEM + 10% FBS + 1% Antibiotics |
| Pancreas | BxPc-3 | Adenocarcinoma | RPMI1640 + 10% FBS + 1% Antibiotics |
| Breast | MDA-M8-231 | Adenocarcinoma | RPMI1640 + 10% FBS + 1% Antibiotics |
| Biliary | SNU-1079 | biliary tract cancer, | RPMI1640 + 10% FBS + 1% Antibiotics |
| tract | | Intrahepatic duct | |
| Skin | A2058 | Melanoma | DMEM + 10% FBS + 1% Antibiotics |
| Brain | U-251 MG | malignant | MEM + 10% FBS + 1% Antibiotics |
| | | glioblastoma | |
| Brain | U-87 MG | Likely | MEM + 10% FBS + 1% Antibiotics |
| | | Glioblastoma | |

B. Transfection with Transfection Reagent (Lipofectamine RNAiMAX)

Depending on the characteristics of cells and a plate size, an appropriate number of cells were seeded, and the medium was replaced after 24 hours of culture (10% FBS, without antibiotics). ASO was added in FBS-free medium in accordance with a concentration, and transfection reagents were also added in the FBS-free medium.

A mixture including ASO and a mixture including the transfection reagent were mixed in 1:1 and reacted at room temperature for 5 minutes. The mixture was dispensed into medium-replaced cell lines and cultured in an incubator (37° C., 5% CO₂) for 24 or 48 hours, depending on the purpose of the test.

Cell seeding and transfection reagents are as shown in Table 3 below:

TABLE 3

| | Cell | Lipofectamine | Overall |
|---|---|---|---|
| Plate | count | RNAiMAX | volume |
| 6 wells | 200,000-300,000 | 7.5 μl | 2 ml |
| 96 wells | 5,000-10,000 | 0.3 μl | 100 μl |

C. Transfection without Transfection Reagents

Depending on the characteristics of cells and a plate size, an appropriate number of cells were seeded and cultured for 24 hours.

ASO was added in a medium including 10% FBS in accordance with a concentration.

The mixture including ASO was dispensed into the cell-seeded plates and then cultured in an incubator (37° C., 5% CO₂) for 24 or 48 hours, depending on the purpose of the test.

2. Quantitative Analysis of TGF-β2 mRNA
a. Extraction and Quantitative Analysis of RNA

After 24 or 48 hours of transfection, all culture solutions were removed, RNA was extracted using an RNA preparation kit (Rneasy Plus Mini kit, Qiagen, Cat #74136), and then RNA was quantified by an absorbance measurement method using a micro-volume plate (Take 3, Biotek) and a multi-plate reader (Synergy H1, Biotek).

B. Synthesis of cDNA

cDNA was synthesized with 2 g of the extracted RNA using a cDNA Synthesis kit (RevertAid First Strand cDNA Synthesis kit, Thermo Sceientific, K1622). Synthesis was carried out under synthesis conditions according to the manufacturer's instructions, and after completion of PCR, the cDNA was diluted with distilled water (DW) to be 20 ng/l.

Real-time PCR was performed for the quantitative analysis of TGF-β2 mRNA. Human SRSF9 was used as the reference gene.

The mixture was prepared in a composition and conditions as shown in Tables 4 to 6 below, and PCR was carried out.

TABLE 4
Composition of PCR mixture

| | Volume per one sample |
|---|---|
| | (ul) |
| DW (ul) | 2 |
| cDNA (20 ng/ul) | 2.5 |
| Primer (F + R, 10 pmole/ul) (ul) | 0.5 |
| SYBR Green (ul) | 5 |
| Total volume (ul) | 10 |

TABLE 5
Primer Information

| | | |
|---|---|---|
| Human SRSF9 | F | TGTGCAGAAGGATGGAGT |
| | R | CTGGTGCTTCTCTCAGGATA |
| Human TGF-β2 | F | CAGCACACTCGATATGGACCA |
| | R | CCTCGGGCTCAGGATAGTCT |

TABLE 6
PCR Conditions

| | | Temperature | | | Data |
|---|---|---|---|---|---|
| Stage | Step | (° C.) | Time | Cycle | collection |
| Hold stage | Step 1 | 50 | 2 min | 1 | |
| | Step 2 | 96 | 10 min | | |
| PCR stage | Step 1 | 96 | 15 sec | 40 | |
| | Step 2 | 60 | 1 min | | Data collection |
| Melt Curve | Step 1 | 96 | 15 sec | 1 | |
| stage | Step 2 | 60 | 1 min | | Data collection |
| | Step 3 | 96 | 15 sec | | |
| | (Dissociation) | | | | |

### 3. Quantitative Analysis on Expression of TGF-β2 Protein (ELISA)

An appropriate number of cells were seeded, and ASO was transfected after 24 hours.

48 hours after transfection, the supernatant was collected and centrifuged (13,000 rpm, 1 minute).

Only the supernatant was collected to be subjected to quantitative analysis according to the manufacturer's instructions using a TGF-β2 ELISA kit (R&D).

### 4. Preparation of ASO Substances

The ASO substance in Table 1 above was prepared by known methods that are commonly used (e.g., referring to a method described in the paper S. L. Beaucage and R. P. Iyer, Tetrahedron, 1993, 49, 6123; or S. L. Beaucage and R. P. Iyer, Tetrahedron, 1992, 48, 2223):

Experimental Example 5. Results of treating A549 cell lines with 20 nM ASO

After treating the cell line A549 with ASO at a concentration of 20 nM in the presence of the transfection reagent, the changes in the mRNA level of TGF-β2 were compared by each candidate substance.

The culture medium for cell line A549 (lung carcinoma) was RPMI1640+10% FBS+1% antibiotics.

2×10⁵ cells/2 ml cells per well were seeded on a 6-well plate.

Transfection: After 24 hours of cell seeding, the medium was replaced (after removal of the existing culture solution, 1.8 ml of media including 10% FBS without antibiotics was added), the ASO to be tested was added in the plain media to be 20 times the treatment concentration (400 nM), and the transfection reagent (Lipofectamine RNAiMAX) was also added in the plain media to reach a concentration of 7.5 ul/100 ul. The mixture including pre-diluted ASO and the mixture including the transfection reagent were mixed in 1:1 and reacted at room temperature for 5 minutes (here, the concentration of ASO is 200 nM). To reach a final concentration for the mixture of the reacted ASO and transfection reagent to 20 nM, the mixture was dispensed by 200 ul to the medium-replaced cell lines, followed by culture for 24 hours (CO₂ incubator (37° C., 5.0% CO₂)).

Quantitative analysis of TGF-β2 mRNA:

RNA extraction and quantification: After 24 hours of transfection, all culture solutions were removed, and then RNA was extracted using an RNA preparation kit (Rneasy Plus Mini Kit, Qiagen, Cat #74136). After RNA extraction, RNA was quantified at OD_{260 nm} by an absorbance measurement method using a micro-volume plate (Take 3, Biotek) and a multi-plate reader (Synergy H1, Biotek).

cDNA synthesis: cDNA was synthesized with 2 μg of the extracted RNA using a cDNA synthesis kit (RevertAid First Strand cDNA Synthesis Kit, Thermo Scientific, K1622). The synthesis conditions were followed by the manufacturer's instruction, and the cDNA was diluted with distilled water (DW) to be 20 ng/ul after completion of PCR.

Running of real-time PCR for quantitative analysis of TGF-β2 mRNA: Using human SRSF9 as the reference gene, a PCR mixture was prepared in the same composition and conditions as in Tables 4-6 above, and PCR was performed.

The results were identified by calculating an mRNA expression inhibition rate (%) of human TGF-β2 with a relative ratio to untreated cells (cell lines solely treated with the transfection reagent without ASO).

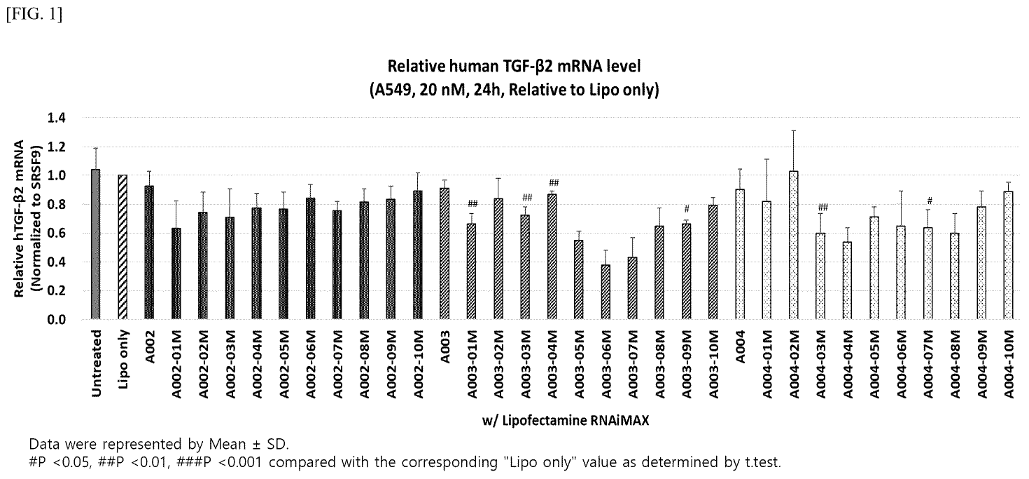

FIG. 1 shows identification of changes in human TGF-β2 mRNA levels upon treatment of 20 nM ASO (along with the transfection reagent) to A549.

The result of the experiment is as shown in Table 7 below:

TABLE 7

| ASO Modification | | Motif | Concentration | Inhibition % |
|---|---|---|---|---|
| A002 | — | — | 20 nM | 7.6 |
| A002-01M | MOE | 4-12-4 | 20 nM | 36.7 |
| A002-02M | MOE | 3-14-3 | 20 nM | 25.9 |
| A002-03M | MOE | 4-14-2 | 20 nM | 29.2 |
| A002-04M | MOE | 5-10-5 | 20 nM | 22.6 |
| A002-05M | MOE | 5-11-4 | 20 nM | 23.3 |
| A002-06M | MOE | 5-12-3 | 20 nM | 15.7 |
| A002-07M | MOE | 5-13-2 | 20 nM | 24.5 |
| A002-08M | MOE | 4-11-5 | 20 nM | 18.4 |
| A002-09M | MOE | 3-12-5 | 20 nM | 16.4 |
| A002-10M | MOE | 2-13-5 | 20 nM | 11.0 |
| A003 | — | — | 20 nM | 8.9 |
| A003-01M | MOE | 4-12-4 | 20 nM | 33.7 |
| A003-02M | MOE | 3-14-3 | 20 nM | 16.1 |
| A003-03M | MOE | 4-14-2 | 20 nM | 27.7 |
| A003-04M | MOE | 5-10-5 | 20 nM | 13.3 |
| A003-05M | MOE | 5-11-4 | 20 nM | 45.2 |
| A003-06M | MOE | 5-12-3 | 20 nM | 62.1 |
| A003-07M | MOE | 5-13-2 | 20 nM | 57.0 |
| A003-08M | MOE | 4-11-5 | 20 nM | 35.2 |
| A003-09M | MOE | 3-12-5 | 20 nM | 33.8 |
| A003-10M | MOE | 2-13-5 | 20 nM | 20.9 |
| A004 | — | — | 20 nM | 9.8 |
| A004-01M | MOE | 4-12-4 | 20 nM | 18.0 |
| A004-02M | MOE | 3-14-3 | 20 nM | −2.8 |
| A004-03M | MOE | 4-14-2 | 20 nM | 40.2 |
| A004-04M | MOE | 5-10-5 | 20 nM | 46.4 |
| A004-05M | MOE | 5-11-4 | 20 nM | 28.7 |
| A004-06M | MOE | 5-12-3 | 20 nM | 35.3 |
| A004-07M | MOE | 5-13-2 | 20 nM | 36.2 |
| A004-08M | MOE | 4-11-5 | 20 nM | 40.1 |
| A004-09M | MOE | 3-12-5 | 20 nM | 21.7 |
| A004-10M | MOE | 2-13-5 | 20 nM | 11.2 |

Experimental Example 6. Identification of changes in human TGF-β2 mRNA levels after treating A549 and PANC-1 cell lines with 1 uM ASO (free uptake environment)

For A549 and PANC-1 cell lines, each ASO was treated at a concentration of 1 μM in the absence of transfection reagents (free uptake environment), and changes in mRNA levels of TGF-β2 after 24 hours was compared.

### Experimental Method:

The culture medium RPMI 1640+10% FBS+1% antibiotics was used for cell line A549 (lung carcinoma), and the culture medium DMEM+10% FBS+1% antibiotics was used for cell line Panc-1 (pancreas epithelioid carcinoma).

(2-2.5)×10⁵ cells/1.8 ml of cells per well were seeded in a 6-well plate.

### Transfection:

Culture was performed for 24 hours after cell seeding.

ASO was added in culture medium including 10% FBS to reach a concentration (10 uM) 10 times higher than that to be finally treated.

The mixture including ASO was dispensed by 200 μl to reach a final concentration of 1 uM on a cell-seeded plate, followed by culture for 24 hours (CO₂ incubator (37° C., 5.0% CO₂)).

Quantitative analysis of TGF-β2 mRNA:

RNA extraction and quantification: After 24 hours of transfection, all culture solutions were removed, and RNA was extracted using an RNA preparation kit (Rneasy Plus Mini Kit, Qiagen, Cat #74136). After RNA extraction, the concentration was calculated at OD_{260 nm} by an absorbance measurement method using a micro-volume plate (Take 3, Biotek) and a multi-plate reader (Synergy H1, Biotek).

cDNA synthesis: cDNA was synthesized with 2 g of the extracted RNA using a cDNA synthesis kit (RevertAid First Strand cDNA Synthesis Kit, Thermo Scientific, K1622). The synthesized cDNA was diluted with distilled water (DW) to be 20 ng/ul.

Running of real-time PCR for quantitative analysis of TGF-β2 mRNA: human SRSF9 was used as the reference gene, and then the PCR mixture was prepared in the same composition and conditions as in Tables 4-6 to perform PCR.

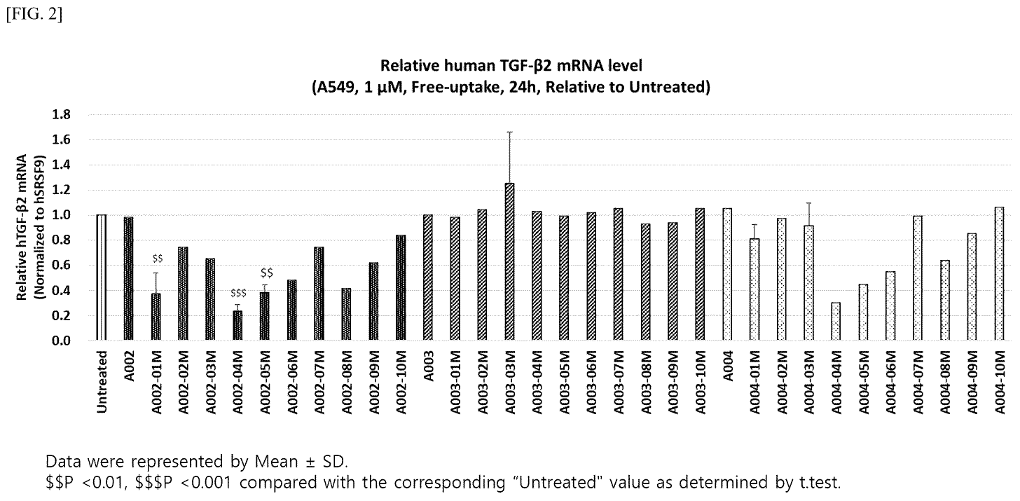

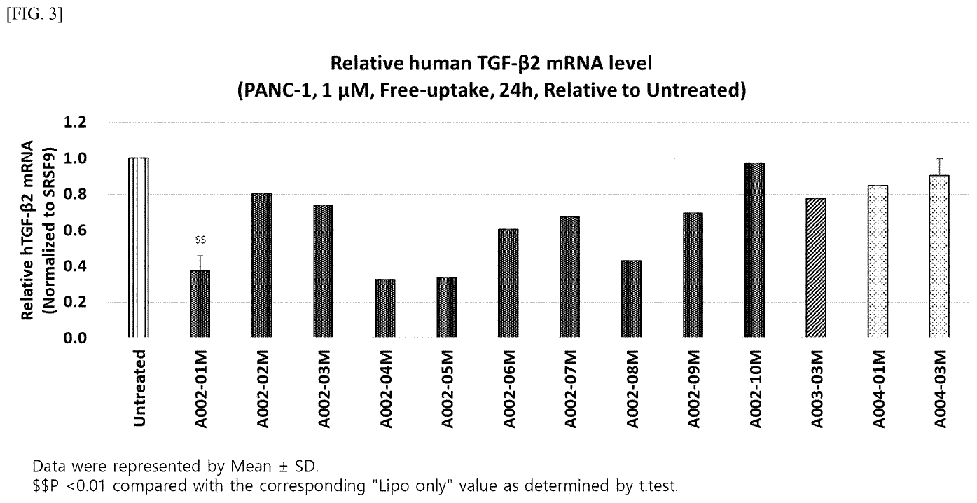

The results were checked by calculating an mRNA expression inhibition rate (%) of human TGF-β2 with a relative ratio to untreated cells (cell lines solely treated with transfection reagents without ASO), wherein the results for the A549 cell line are shown in Table 8 and FIG. 2 below, and the results for the PANC-1 cell line are shown in Table 9 and FIG. 3 below.

TABLE 8

| | | |
|---|---|---|
| ASO Modification | Motif | Inhibition % |

| | | | |
|---|---|---|---|
| A002 | − | − | 2 |
| A002-01M | MOE | 4-12-4 | 63 |
| A002-02M | MOE | 3-14-3 | 26 |
| A002-03M | MOE | 4-14-2 | 35 |
| A002-04M | MOE | 5-10-5 | 76 |
| A002-05M | MOE | 5-11-4 | 62 |
| A002-06M | MOE | 5-12-3 | 52 |
| A002-07M | MOE | 5-13-2 | 26 |
| A002-08M | MOE | 4-11-5 | 58 |
| A002-09M | MOE | 3-12-5 | 38 |
| A002-10M | MOE | 2-13-5 | 16 |
| A003 | − | − | 0 |
| A003-01M | MOE | 4-12-4 | 2 |
| A003-02M | MOE | 3-14-3 | −4 |
| A003-03M | MOE | 4-14-2 | −25 |
| A003-04M | MOE | 5-10-5 | −3 |
| A003-05M | MOE | 5-11-4 | 1 |
| A003-06M | MOE | 5-12-3 | −2 |
| A003-07M | MOE | 5-13-2 | −5 |
| A003-08M | MOE | 4-11-5 | 7 |
| A003-09M | MOE | 3-12-5 | 6 |
| A003-10M | MOE | 2-13-5 | −5 |
| A004 | − | − | −5 |
| A004-01M | MOE | 4-12-4 | 19 |
| A004-02M | MOE | 3-14-3 | 3 |
| A004-03M | MOE | 4-14-2 | 9 |
| A004-04M | MOE | 5-10-5 | 70 |
| A004-05M | MOE | 5-11-4 | 55 |
| A004-06M | MOE | 5-12-3 | 45 |
| A004-07M | MOE | 5-13-2 | 1 |
| A004-08M | MOE | 4-11-5 | 36 |
| A004-09M | MOE | 3-12-5 | 15 |
| A004-10M | MOE | 2-13-5 | −6 |

TABLE 9

| | | |
|---|---|---|
| ASO Modification | Motif | % inhibition |

| | | | |
|---|---|---|---|
| A002-01M | MOE | 4-12-4 | 62 |
| A002-02M | MOE | 3-14-3 | 19.7 |
| A002-03M | MOE | 4-14-2 | 26.5 |
| A002-04M | MOE | 5-10-5 | 67.5 |
| A002-05M | MOE | 5-11-4 | 66.5 |
| A002-06M | MOE | 5-12-3 | 39.7 |
| A002-07M | MOE | 5-13-2 | 32.7 |
| A002-08M | MOE | 4-11-5 | 57 |
| A002-09M | MOE | 3-12-5 | 30.4 |
| A002-10M | MOE | 2-13-5 | 2.7 |
| A003-03M | MOE | 4-14-2 | 22.3 |
| A004-01M | MOE | 4-12-4 | 17.5 |
| A004-03M | MOE | 4-14-2 | 8.9 |

Experimental Example 7: Comparison of free uptakes in the A549 cell line using 5′-fluorescein amidite (FAM) ASO

Free uptakes (absorption under transfection reagent-free environment) were compared in A549, PANC-1, and A2058 cell lines using 5′-fluorescein amidite (FAM)-labeled ASO.

### Experimental Method:

Culture medium RPMI1640+10% FBS+1% antibiotics was used for cell line A549 (lung carcinoma), and culture medium DMEM+10% FBS+1% antibiotics was used for cell lines PANC-1 (pancreas epithelioid carcinoma) and A2058 (melanoma).

### Cell Seeding:

4×10⁵ cells/500 μμl of cells per well were seeded in a 4-chamber slide.

Transfection:

The ASO to be tested was dispensed into each well by 200 μl to reach a concentration of 1 μM in DMEM medium including 10% FBS and 1% antibiotics, followed by culture for 24 hours (CO₂ incubator 37° C., 5.0% CO₂).

### FACS Analysis

Cells were detached by treating trypsin-EDTA and washed with PBS. After the cells were suspended with PBS, fluorescence-activated cell sorter (FACS) analysis was performed. Mean fluorescence intensity (MFI) was checked, and the relative content of the FAM-labeled ASO of each cell was compared.

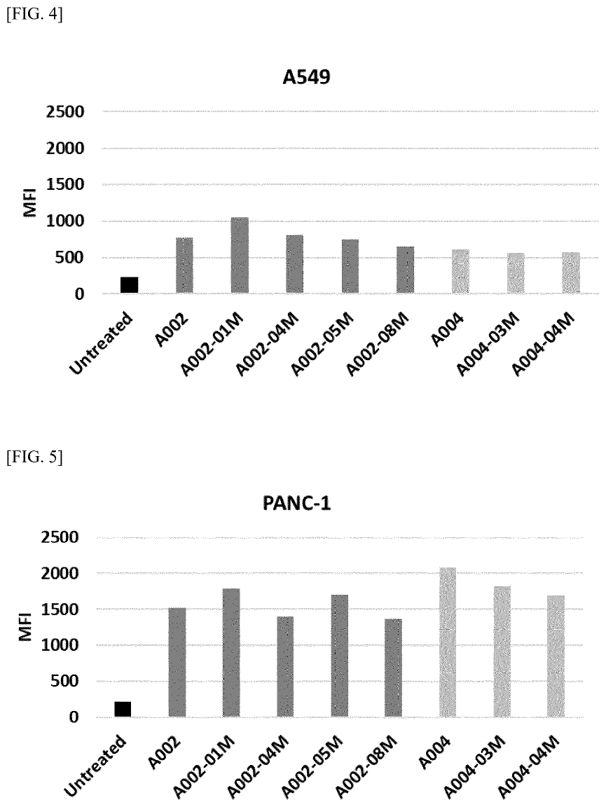

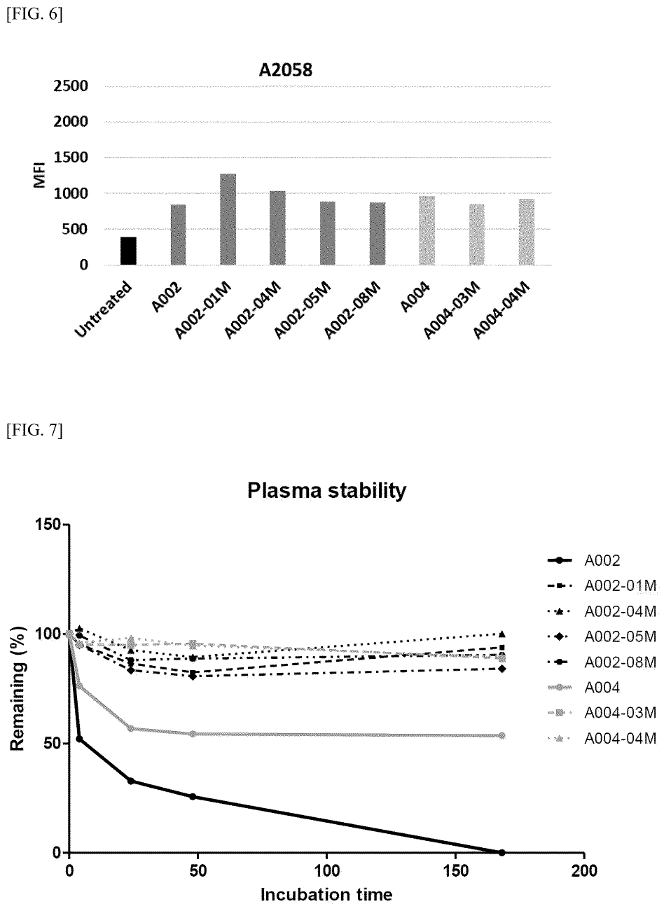

The results of MFI measurements for the A549 cell line, the PAC-1 cell line, and the A2058 cell line are shown in FIGS. 4, 5, and 6, respectively.

As a result of the experiment, it was found that all ASOs used in the test were properly absorbed into the cells.

Experimental Example 8: Measurement of stability after spiking ASO into human plasma

ASO was spiked into human plasma and cultured for 7 days, and the amount of ASO remaining was analyzed.

### Experimental Method:

Spiking was performed to make ASO in the human plasma reach a final concentration of 5 uM and incubated for 7 days in a 37° C. incubator with rotation at 40 rpm. Plasma was sampled at different times (day 0, 4 hours, day 1, day 2, and day 7), and the amount of residual ASO was analyzed by HPLC. A proportion of the remaining ASO amount was calculated based on the amount of ASO detected at hour 0.

The result of the experiment is shown in FIG. 7.

The ASO substance according to the present disclosure had significantly increased stability compared to naked ASO (A002 and A004) and was found to remain in the plasma by more than 80% even after 7 days.

Experimental Example 9: Identification of changes in levels of mRNA and protein release after treatment of ASO by concentration in the presence of the transfection reagent for A549 cell lines (5-160 nM)

After treating A549 cell lines with ASO by concentration (0 nM, 5 nM, 20 nM, 80 nM) in the presence of the transfection reagents, changes in mRNA levels and protein release levels of TGF-β2 were checked.

### Experimental Method:

The culture medium RPMI1640+10% FBS+1% antibiotics was used for cell line A549 (lung carcinoma).

2×10⁵ cells/2 ml cells per well were seeded in a 6-well plate.

### Transfection:

24 hours after cell seeding, the medium was replaced (after removal of the existing culture solution, 1.8 ml of medium including 10% FBS without antibiotics was added). The ASO to be tested was diluted in plain media to be 20 times the concentration to be treated.

The transfection reagent (Lipofectamine RNAiMAX) was also diluted with plain media to reach a concentration of 7.5 l/100 l.

A mixture including pre-diluted ASO and a mixture including the transfection reagent were mixed in a volume ratio of 1:1 and reacted at room temperature for 5 minutes.

To reach the preset final concentration, a mixture of the reacted ASO and transfection reagent was dispensed by 200 ul into the medium-replaced cell line, followed by culture for 24 hours. (CO₂ incubator (37° C., 5.0% CO₂))

The medium was replaced with 1 ml of medium which includes 1% FBS without antibiotics, followed by culture for 48 hours. (CO₂ incubator (37° C., 5.0% CO₂))

Quantitative Analysis of TGF-β2 mRNA:

RNA extraction and quantification: After 72 hours of transfection, all culture solutions were removed, and RNA was extracted using an RNA preparation kit (Rneasy Plus Mini Kit, Qiagen, Cat #74136). After RNA extraction, the concentration was calculated at OD_{260 nm} by an absorbance measurement method using a micro-volume plate (Take 3, Biotek) and a multi-plate reader (Synergy H1, Biotek).

cDNA synthesis: cDNA was synthesized with 2 g of the extracted RNA using a cDNA synthesis kit (RevertAid First Strand cDNA Synthesis Kit, Thermo Scientific, K1622). The synthesized cDNA was diluted to be 20 ng/l using distilled water (DW).

Running of real-time PCR for TGF-β2 mRNA quantitative analysis: Human SRSF9 was used as the reference gene. The PCR mixture was prepared in the same composition and conditions as shown in Tables 4 to 6 above, and PCR was carried out.

### Quantitative Analysis on TGF-β2 Protein Expression (ELISA)

Separately collected culture solution was centrifuged, only the supernatant was collected, and quantitative analysis was conducted according to the manufacturer's instruction using a TGF-β2 ELISA kit (R&D).

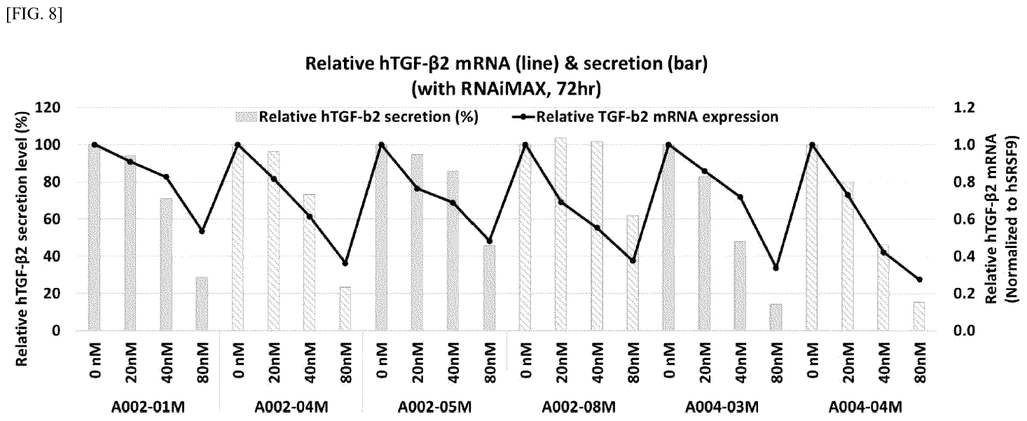

The result of the experiment is shown in FIG. 8. As a result of the experiment, it was determined that ASO according to the present disclosure decreases human TGF 32 mRNA levels and secretion levels in a concentration-dependent manner.

Experimental Example 10: Identification of human TGF-β2 mRNA inhibition after treatment of ASO by concentration in absence of transfection reagent (free uptake condition)

After treating ASO to A549, PANC-1, and A2058 cell lines by concentration (3.9 nM-4000 nM) under free uptake conditions without the transfection reagents, changes in mRNA levels of human TGF-β2 compared to untreated cells were checked.

### Experimental Method:

The culture medium RPMI1640+10% FBS+1% antibiotics was used for cell line A549 (lung carcinoma), and the culture medium DMEM+10% FBS+1% antibiotics was used for cell lines PANC-1 (pancreas epithelioid carcinoma) and A2058 (melanoma).

(2˜2.5)×10⁵ cells/2 ml of cells per well were seeded in a 6-well plate.

### Transfection:

24 hours after cell seeding, the medium was replaced (after removal of the existing culture solution, 1.8 ml of medium including 10% FBS without antibiotics was added). The ASO to be tested was diluted in plain media to become 10 times the concentration to be treated.

To reach the preset final concentration, the mixture including pre-diluted ASO was dispensed by 200 μl to the medium-replaced cell line, followed by culture for 48 hours. (CO₂ incubator (37° C., 5.0% CO₂))

Quantitative Analysis of TGF-β2 mRNA:

RNA extraction and quantification: After 48 hours of transfection, all culture solutions were removed, and RNA was extracted using an RNA preparation kit (Rneasy Plus Mini Kit, Qiagen, Cat #74136). After RNA extraction, the concentration was calculated at OD_{260 nm} by an absorbance measurement method using a micro-volume plate (Take 3, Biotek) and a multi-plate reader (Synergy H1, Biotek).

cDNA synthesis: cDNA was synthesized with 2 g of the extracted RNA using a cDNA synthesis kit (RevertAid First Strand cDNA Synthesis Kit, Thermo Scientific, K1622). The synthesized cDNA was diluted to be 20 ng/l using distilled water (DW).

Running of real-time PCR for TGF-β2 mRNA quantitative analysis: Human SRSF9 was used as the reference gene. The PCR mixture was prepared in the same composition and conditions as shown in Tables 4 to 6 above, and PCR was carried out.

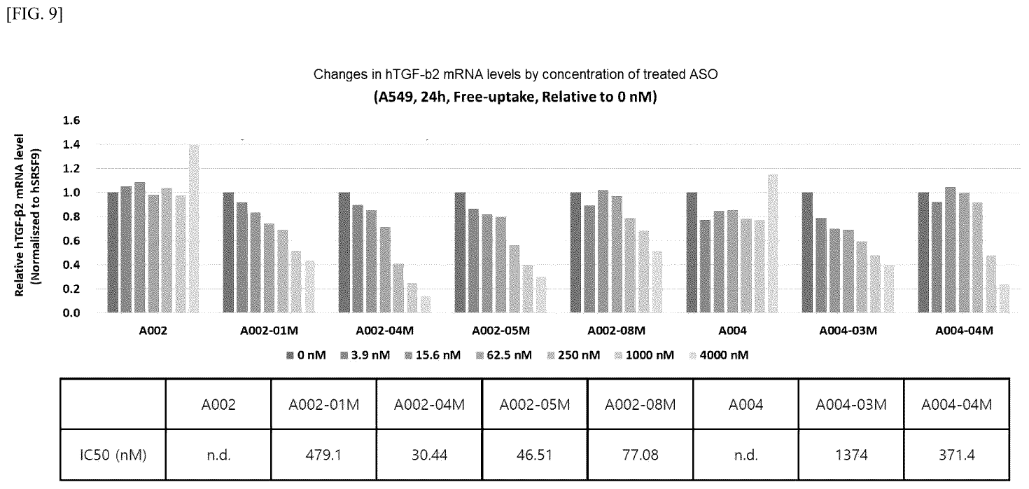

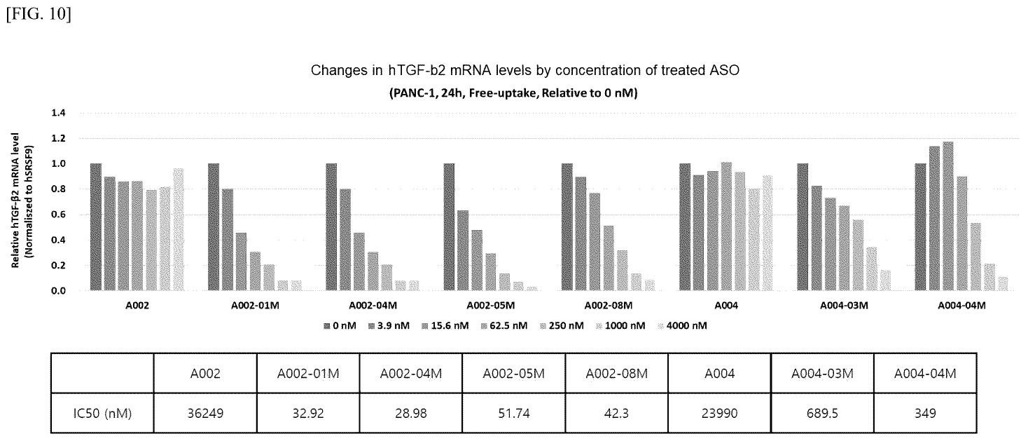

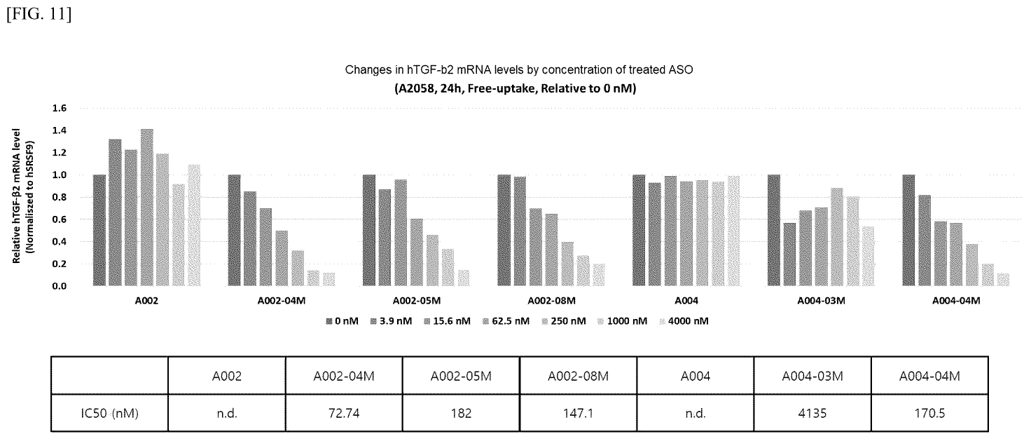

The result of the experiment for the A549 cell line, the PAC-1 cell line, and the A2058 cell line are shown in FIGS. 9, 10, and 11, respectively.

As a result of the experiment, A002-04M, A002-05M, and A002-08M had particularly superior effects of inhibiting TGF-β2 mRNA for the A549 cell line, A002-01M, A002-04M, A002-05M, and A002-08M had particularly excellent effects of inhibiting TGF-32 mRNA for the PANC-1 cell line, and A002-04M, A002-05M, A002-08M, and A004-04M had particularly superior effects of inhibiting TGF-β2 mRNA for the A2058 cell line.

Experimental Example 11: Identification of changes in human TGF-β2 mRNA levels after treatment of ASO in the absence of the transfection reagent (free uptake condition) for cell lines from various carcinomas

Cell lines from various carcinomas were treated with ASO, and a reduction in TGF-02 mRNA levels was identified after 24 or 72 hours.

### Experimental Method:

The cell line of an experimental subject and medium used are the same as shown in Table 2 above.

(2˜2.5)×10⁵ cells/2 ml of cells per well were seeded in a 6-well plate.

### Transfection:

24 hours after cell seeding, 1.8 ml of medium including 1% FBS was added.

The ASO to be tested was diluted in plain media to be 10 times the concentration to be treated.

To reach the preset final concentration, the mixture including pre-diluted ASO was dispensed by 200 μl to the medium-replaced cell line, followed by culture 24 or 72 hours. (CO₂ incubator (37° C., 5.0% CO₂))

Quantitative Analysis of TGF-β2 mRNA:

RNA extraction and quantification: 24 hours after transfection, all culture solutions were removed, and RNA was extracted using an RNA preparation kit (Rneasy Plus Mini Kit, Qiagen, Cat #74136). After RNA extraction, the concentration was calculated at OD_{260 nm} by an absorbance measurement method using a micro-volume plate (Take 3, Biotek) and a multi-plate reader (Synergy H1, Biotek).

cDNA synthesis: cDNA was synthesized with 2 g of the extracted RNA using a cDNA synthesis kit (RevertAid First Strand cDNA Synthesis Kit, Thermo Scientific, K1622). The synthesized cDNA was diluted to be 20 ng/l using distilled water (DW).

Running of real-time PCR for TGF-β2 mRNA quantitative analysis: Human SRSF9 was used as the reference gene. The PCR mixture was prepared in the same composition and conditions as shown in Tables 4 to 6 above, and PCR was carried out.

The mRNA inhibition rate (%) of human TGF-β2 was calculated with a relative ratio to untreated cells (cell lines solely treated with the transfection reagents without ASO).

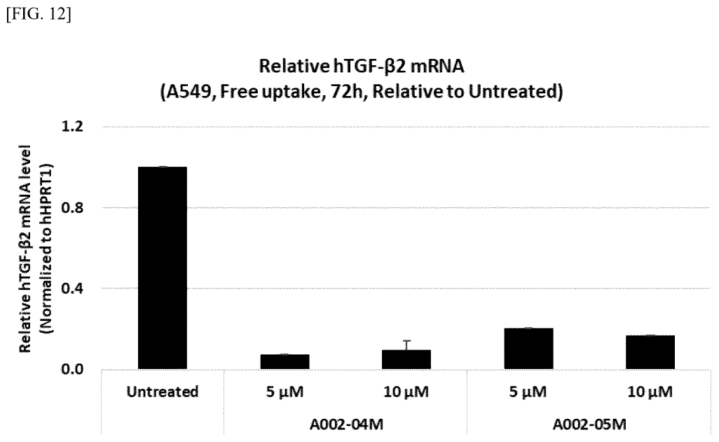

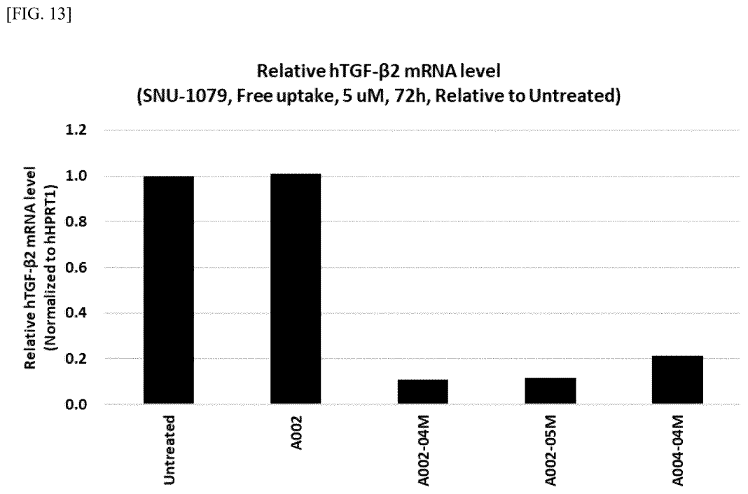

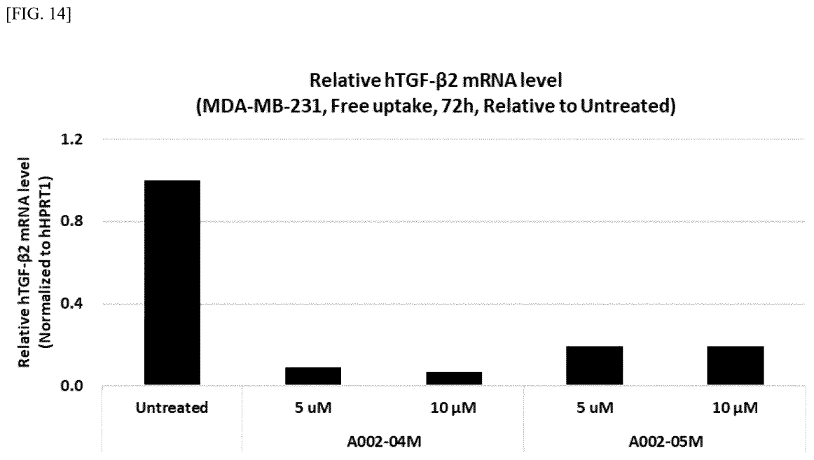

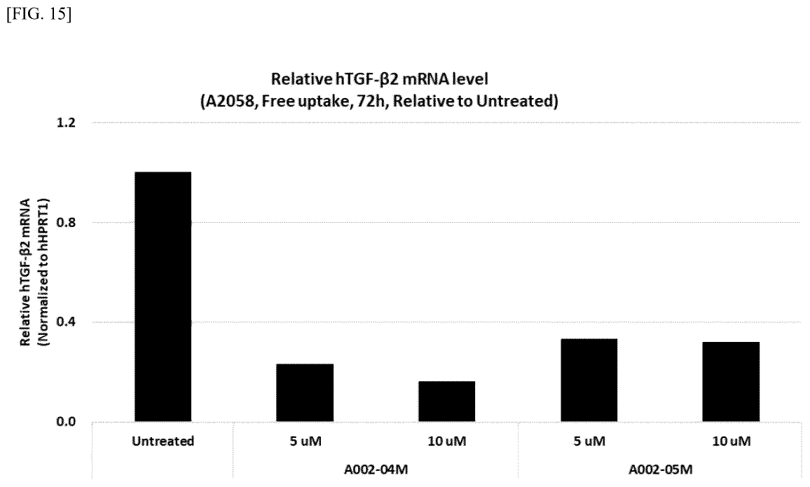

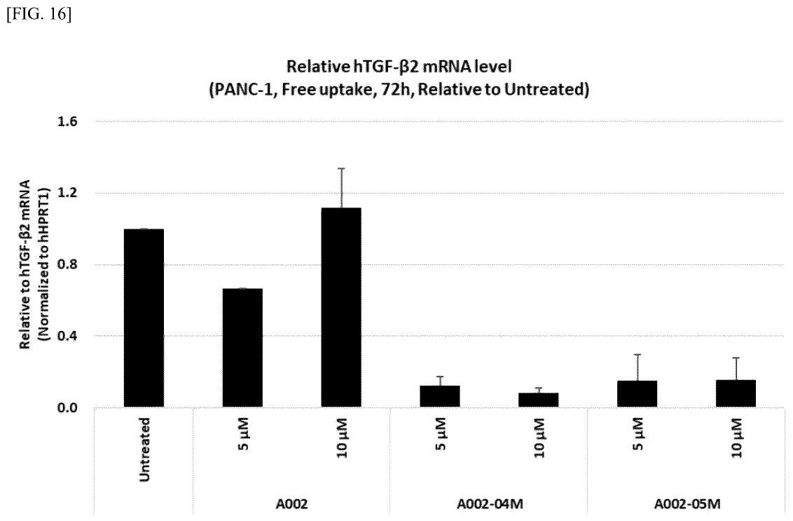

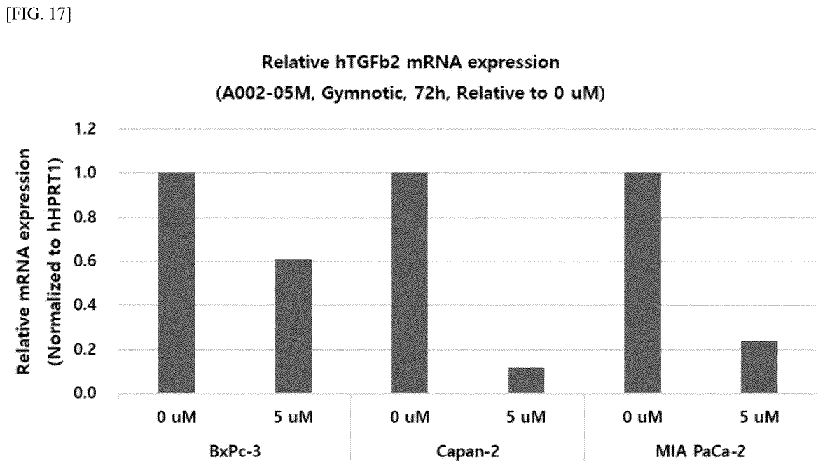

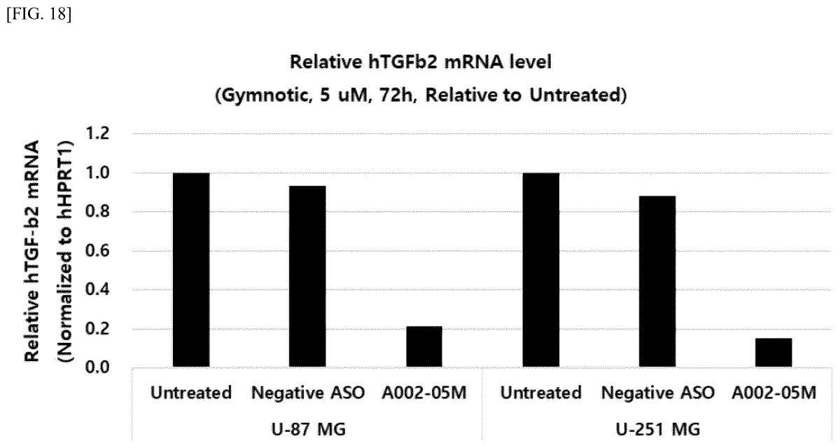

The results of the experiment for each cell line is shown in FIGS. 12 to 18.

As a result of the experiment, it was found that hTGF-β2 mRNA levels were remarkably reduced in various carcinomas (lung cancer, cholangiocarcinoma, triple negative breast cancer, melanoma, pancreatic cancer, and glioblastoma) by A002-04M or A002-05M.

Experimental Example 12: Evaluation on anticancer efficacy in humanized xenograft mouse models transplanted with melanoma cell line A2058

### Humanized A2058 Xenograft Model:

Mouse: NSG-b2m immunodeficient mice, females 5-7 weeks old, 6 mice/group

After the mice are obtained, the mice were tamed for a week.

Human peripheral blood mononuclear cells (PBMCs: Zenbio, Cat #SER-PBMC-200-F) were transplanted by intravenous (i.v.) administration with 1×10⁷ cells/mouse.

5 days after PBMC transplantation, the A2058 cell line was transplanted by subcutaneous administration with 2×10⁶ cells/mouse.

5 days after transplantation of the A2058 cell line, administration was initiated through intraperitoneal injection of ASO according to the present disclosure:

Treatment: 3 times a week in a dosage of 30 mg/kg and an administration volume of 10 ml/kg (i.e., the concentration of the injection solution is 30 mg/10 ml ASO)

Observation: Measurement of a body weight and a tumor volume twice a week

The overall experimental design for sets A, B, and C below is similar, but separated by each donor of the transplanted PBMCs.

The experimental design of each group for sets A, B, and C is as shown in Tables 10 to 12 below:

TABLE 10
Set A

| Group | N | PBMC | Agent | Dose | Route | Injection interval |
|---|---|---|---|---|---|---|
| 1 | 6 | — | PBS | — | IP | TIW |
| 2 | 6 | 1 × 10⁷ | PBS | — | IP | TIW |
| 3 | 6 | 1 × 10⁷ | A002-04M | 30 mpk | IP | TIW |
| 4 | 6 | 1 × 10⁷ | A002-05M | 30 mpk | IP | TIW |

TIW = three times per week/ip = intraperitoneal

TABLE 11
Set B

| | | | | | | Injection |
|---|---|---|---|---|---|---|
| Group | N | PBMC | Agent | Dose | Route | interval |
| 1 | 6 | — | PBS | — | IP | TIW |
| 2 | 6 | 1 × 10⁷ | PBS | — | IP | TIW |
| 3 | 6 | 1 × 10⁷ | A002-05M | 30 mpk | IP | TIW |
| 4 | 6 | 1 × 10⁷ | A002-08M | 30 mpk | IP | TIW |

TABLE 12
Set C

| | | | | | | Injection |
|---|---|---|---|---|---|---|
| Group | N | PBMC | Agent | Dose | Route | interval |
| 1 | 6 | — | PBS | — | IP | TIW |
| 2 | 6 | 1 × 10⁷ | PBS | — | IP | TIW |
| 3 | 6 | 1 × 10⁷ | A002-04M | 30 mpk | IP | TIW |
| 4 | 6 | 1 × 10⁷ | A002-05M | 30 mpk | IP | TIW |
| 5 | 6 | 1 × 10⁷ | A002-08M | 30 mpk | IP | TIW |

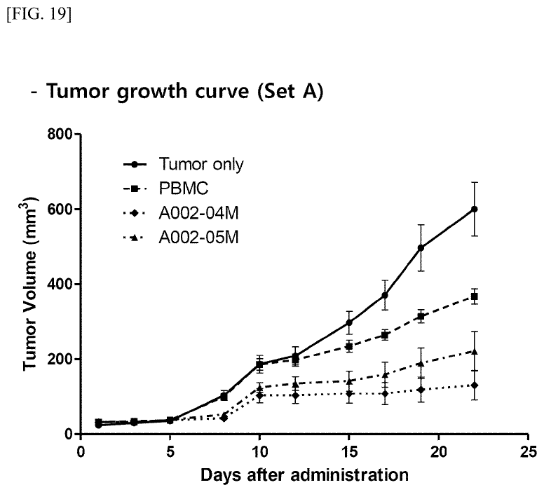

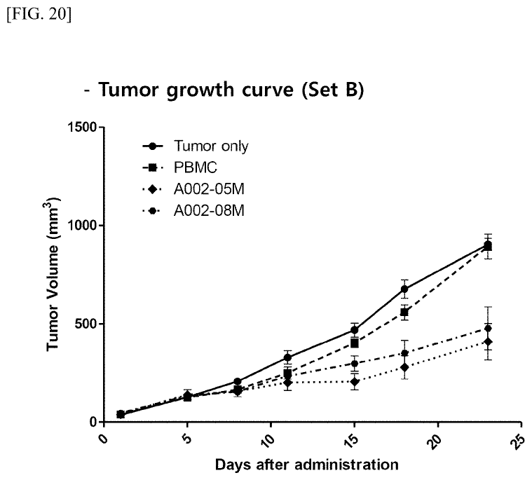

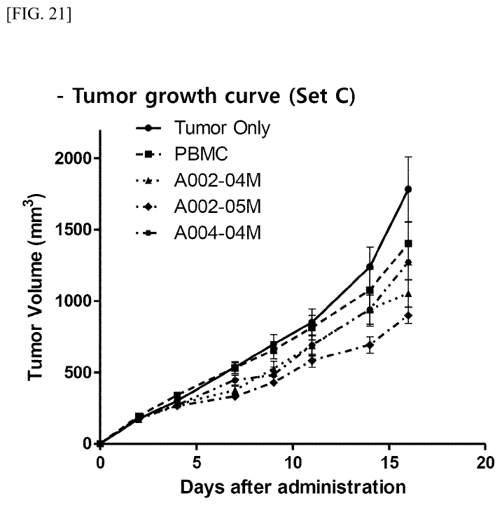

The tumor growth curve for each set is shown in FIG. 19 to FIG. 21.

The tumor growth inhibition rate (IR) for each set is shown in Tables 13 to 15 below:

TABLE 13
Tumor growth inhibition rate (IR) (%) (Set A)

| | | G2 (PBMC) | G3 | G4 |
|---|---|---|---|---|
| Administered substances | | PBS | A002-04M | A002-05M |
| Dosage (mg/kg) | | — | 30 | 30 |
| Usage | | — | IP, TIW | IP, TIW |
| Days after | Day 1 | 0.00 | 0.6 | 0 |
| administration | Day 3 | 0.00 | 4.7 | 4.3 |
| | Day 5 | 0.00 | 2.2 | −0.7 |
| | Day 8 | 0.00 | 57.4 | 47.3 |
| | Day 10 | 0.00 | 44.6** | 32.8* |
| | Day 12 | 0.00 | 47.6** | 31.9* |
| | Day 15 | 0.00 | 53.8** | 39.5** |
| | Day 17 | 0.00 | 58.9** | 39.9* |
| | Day 19 | 0.00 | 62.1** | 39.9* |
| | Day 22 | 0.00 | 64.4** | 39.6* |

TABLE 14
Tumor growth inhibition rate (IR) (%) (Set B)

| | | G2 (PBMC) | G3 | G4 |
|---|---|---|---|---|
| Administered substances | | PBS | A002-05M | A002-08M |
| Dosage (mg/kg) | | — | 30 | 30 |
| Usage | | — | IP, TIW | IP, TIW |
| Days after | Day 1 | 0.0 | −3.8 | −18.9 |
| administration | Day 5 | 0.0 | −11.2 | −7.1 |
| | Day 8 | 0.0 | 5.3 | 6.4 |
| | Day 11 | 0.0 | 19.5 | 6.0 |
| | Day 15 | 0.0 | 48.8 * | 25.8 |
| | Day 18 | 0.0 | 50.1 * | 37.1 |
| | Day 23 | 0.0 | 54.2 ** | 46.6 ** |

TABLE 15
Tumor growth inhibition rate (IR) (%) (Set C)

| | | G2 (PBMC) | G3 | G4 | GS |
|---|---|---|---|---|---|
| Administered substances | | PBS | A002-04M | A002-05M | A004-04M |
| Dosage (mg/kg) | | — | 30 | 30 | 30 |
| Usage | | — | IP, TIW | IP, TIW | IP, TIW |
| Days after | Day 2 | 0.0 | 11.2 | 8.2 | 4.9 |
| administration | Day 4 | 0.0 | 19.6* | 22.1** | 20.8** |
| | Day 7 | 0.0 | 29.5** | 37.8** | 16.4 |
| | Day 9 | 0.0 | 19.6 | 34.6** | 26.5* |
| | Day 11 | 0.0 | 15.4 | 28.3* | 15.1 |
| | Day 14 | 0.0 | 12.6 | 35.6** | 12.4 |
| | Day 16 | 0.0 | 24.9 | 35.8 | 9.2 |

From the result of the experiment, it was determined that the tumor growth inhibition rate upon administration of ASO according to the present disclosure is more superior than the immunotherapeutic effect by PBMC.

### INDUSTRIAL APPLICABILITY

The oligonucleotide of the present disclosure may be used as an anticancer agent by suppressing expression of TGF-02.

### Sequence List Free Text

SEQ ID NO: 1: 5′-GGCGG CATGT CTATT TTGTA-3′
SEQ ID NO: 2: 5′-CGGCA TGTCT ATTTT GTAAA-3′
SEQ ID NO: 3: 5′-GCGGC ATGTC TATTT TGTAA-3′

1. An oligonucleotide which is an antisense oligonucleotide of any one of SEQ ID NOS: 1 to 3,
wherein at least one nucleotide of the oligonucleotide is a nucleotide having a modified nucleoside, and
the modified nucleotide is selected from the group consisting of a 2′-O-methoxyethyl (MOE)-modified nucleotide, a 2′-fluoro (F)-modified nucleotide, a 2′-O-methyl (0-Me)-modified nucleotide, a locked nucleic acid (LNA)-modified nucleotide, an ethylene-bridged nucleic acid (ENA)-modified nucleotide, an (R/S)-constrained ethyl (cET)-modified nucleotide, and a polyalkylene oxide-modified nucleotide.

2. The oligonucleotide of claim 1, wherein one or more nucleotides at a 5′-end or one or more nucleotides at a 3′-end of the oligonucleotide are modified nucleotides.

3. The oligonucleotide of claim 1, wherein one or more nucleotides at a 5′-end and one or more nucleotides at a 3′-end of the oligonucleotide are modified nucleotides.

4. The oligonucleotide of claim 2, wherein the first to n^{th} nucleotides at the 5′-end of the oligonucleotide or the first to m^{th} nucleotides at the 3′-end of the oligonucleotide are the modified nucleotides, wherein n and m are each independently an integer of 1 to 10.

5. The oligonucleotide of claim 3, wherein the first to n^{th} nucleotides at the 5′-end of the oligonucleotide and the first to m^{th} nucleotides at the 3′-end of the oligonucleotide are the modified nucleotides.

6. The oligonucleotide of claim 5, wherein the oligonucleotide further comprises one or more modified nucleotides between the (n+1)^{th} nucleotide at the 5′-end and the (m+1)^{th} nucleotide at the 3′-end of the oligonucleotide.

7. An oligonucleotide which is an antisense oligonucleotide represented by any one of SEQ ID NOS: 5 to 37 in Table 1 below,
wherein nucleotides indicated in bold and underlined letters in Table 1 below are modified nucleotides, and
the modified nucleotide is selected from the group consisting of a 2′-O-methoxyethyl (MOE)-modified nucleotide, a 2′-fluoro (F)-modified nucleotide, a 2′-O-methyl (0-Me)-modified nucleotide, a locked nucleic acid (LNA)-modified nucleotide, an ethylene-bridged nucleic acid (ENA)-modified nucleotide, an (R/S)-constrained ethyl (cET)-modified nucleotide, and a polyalkylene oxide-modified nucleotide:
TABLE 1

| | | NM001135599 | | | | Sequence |
|---|---|---|---|---|---|---|
| No | Substance | Start | End | Modification | Motif | (5′->3′) |
| | A002 | 1695 | 1676 | | | G*G*C*G*G*C*A*T*G*T*C*T*A*T*T*T*T*G*T*A |
| 1 | A002-01M | 1695 | 1676 | MOE | 4-12-4 | G*G*C*G*G*C*A*T*G*T*C*T*A*T*T*T*T*G*T*A |
| 2 | A002-02M | 1695 | 1675 | MOE | 3-14-3 | G*G*C*G*G*C*A*T*G*T*C*T*A*T*T*T*T*G*T*A |
| 3 | A002-03M | 1695 | 1676 | MOE | 4-14-2 | G*G*C*G*G*C*A*T*G*T*C*T*A*T*T*T*T*G*T*A |
| 4 | A002-04M | 1695 | 1676 | MOE | 5-10-5 | G*G*C*G*G*C*A*T*G*T*C*T*A*T*T*T*T*G*T*A |
| 5 | A002-05M | 1695 | 1676 | MOE | 5-11-4 | G*G*C*G*G*C*A*T*G*T*C*T*A*T*T*T*T*G*T*A |
| 6 | A002-06M | 1695 | 1676 | MOE | 5-12-3 | G*G*C*G*G*C*A*T*G*T*C*T*A*T*T*T*T*G*T*A |
| 7 | A002-07M | 1695 | 1676 | MOE | 5-13-2 | G*G*C*G*G*C*A*T*G*T*C*T*A*T*T*T*T*G*T*A |
| 8 | A002-08M | 1695 | 1676 | MOE | 4-11-5 | G*G*C*G*G*C*A*T*G*T*C*T*A*T*T*T*T*G*T*A |
| 9 | A002-09M | 1695 | 1676 | MOE | 3-12-5 | G*G*C*G*G*C*A*T*G*T*C*T*A*T*T*T*T*G*T*A |
| 10 | A002-10M | 1695 | 1676 | MOE | 2-13-5 | G*G*C*G*G*C*A*T*G*T*C*T*A*T*T*T*T*G*T*A |
| | A003 | 1693 | 1674 | | | C*G*G*C*A*T*G*T*C*T*A*T*T*T*T*G*T*A*A*A |
| 11 | A003-01M | 1693 | 1674 | MOE | 4-12-4 | C*G*G*C*A*T*G*T*C*T*A*T*T*T*T*G*T*A*A*A |
| 12 | A003-02M | 1693 | 1674 | MOE | 3-14-3 | C*G*G*C*A*T*G*T*C*T*A*T*T*T*T*G*T*A*A*A |
| 13 | A003-03M | 1693 | 1674 | MOE | 4-14-2 | C*G*G*C*A*T*G*T*C*T*A*T*T*T*T*G*T*A*A*A |
| 14 | A003-04M | 1693 | 1674 | MOE | 5-10-5 | C*G*G*C*A*T*G*T*C*T*A*T*T*T*T*G*T*A*A*A |
| 15 | A003-05M | 1693 | 1674 | MOE | 5-11-4 | C*G*G*C*A*T*G*T*C*T*A*T*T*T*T*G*T*A*A*A |
| 16 | A003-06M | 1693 | 1674 | MOE | 5-12-3 | C*G*G*C*A*T*G*T*C*T*A*T*T*T*T*G*T*A*A*A |
| 17 | A003-07M | 1693 | 1674 | MOE | 5-13-2 | C*G*G*C*A*T*G*T*C*T*A*T*T*T*T*G*T*A*A*A |
| 18 | A003-08M | 1693 | 1674 | MOE | 4-11-5 | C*G*G*C*A*T*G*T*C*T*A*T*T*T*T*G*T*A*A*A |
| 19 | A003-09M | 1693 | 1674 | MOE | 3-12-5 | C*G*G*C*A*T*G*T*C*T*A*T*T*T*T*G*T*A*A*A |
| 20 | A003-10M | 1693 | 1674 | MOE | 2-13-5 | C*G*G*C*A*T*G*T*C*T*A*T*T*T*T*G*T*A*A*A |
| | A004 | 1694 | 1675 | | | G*C*G*G*C*A*T*G*T*C*T*A*T*T*T*T*G*T*A*A |
| 21 | A004-01M | 1694 | 1675 | MOE | 4-12-4 | G*C*G*G*C*A*T*G*T*C*T*A*T*T*T*T*G*T*A*A |
| 22 | A004-02M | 1694 | 1675 | MOE | 3-14-3 | G*C*G*G*C*A*T*G*T*C*T*A*T*T*T*T*G*T*A*A |
| 23 | A004-03M | 1694 | 1675 | MOE | 4-14-2 | G*C*G*G*C*A*T*G*T*C*T*A*T*T*T*T*G*T*A*A |
| 24 | A004-04M | 1694 | 1675 | MOE | 5-10-5 | G*C*G*G*C*A*T*G*T*C*T*A*T*T*T*T*G*T*A*A |
| 25 | A004-05M | 1694 | 1675 | MOE | 5-11-4 | G*C*G*G*C*A*T*G*T*C*T*A*T*T*T*T*G*T*A*A |
| 26 | A004-06M | 1694 | 1675 | MOE | 5-12-3 | G*C*G*G*C*A*T*G*T*C*T*A*T*T*T*T*G*T*A*A |
| 27 | A004-07M | 1694 | 1675 | MOE | 5-13-2 | G*C*G*G*C*A*T*G*T*C*T*A*T*T*T*T*G*T*A*A |
| 28 | A004-08M | 1694 | 1675 | MOE | 4-11-5 | G*C*G*G*C*A*T*G*T*C*T*A*T*T*T*T*G*T*A*A |
| 29 | A004-09M | 1694 | 1675 | MOE | 3-12-5 | G*C*G*G*C*A*T*G*T*C*T*A*T*T*T*T*G*T*A*A |
| 30 | A004-10M | 1694 | 1675 | MOE | 2-13-5 | G*C*G*G*C*A*T*G*T*C*T*A*T*T*T*T*G*T*A*A |

8. The oligonucleotide of claim 7, wherein a modified nucleotide of an oligonucleotide represented by any one of SEQ ID NOS: 5 to 37 in Table 1 is a 2′-O-methoxyethyl (MOE)-modified nucleotide.

9. The oligonucleotide of claim 1, wherein a bond between neighboring nucleosides is either a phosphodiester bond or a phosphothioate bond.

10. The oligonucleotide of claim 1, wherein a bond between neighboring nucleosides is a phosphothioate bond.

11. A method of treating cancer, comprising:
administering a pharmaceutical composition comprising an antisense oligonucleotide of any one of SEQ ID NOS: 1 to 3,
wherein the antisense oligonucleotide is a nucleotide having a modified nucleoside,
wherein the modified nucleotide is selected from the group consisting of a 2′-O-methoxyethyl (MOE)-modified nucleotide, a 2′-fluoro (F)-modified nucleotide, a 2′-O-methyl (O-Me)-modified nucleotide, a locked nucleic acid (LNA)-modified nucleotide, an ethylene-bridged nucleic acid (ENA)-modified nucleotide, an (R/S)-constrained ethyl (cET)-modified nucleotide, and a polyalkylene oxide-modified nucleotide.

12. The method of claim 11, wherein one or more nucleotides at a 5′-end or one or more nucleotides at a 3′-end of the oligonucleotide are modified nucleotides.

13. The method of claim 11, wherein one or more nucleotides at a 5′-end and one or more nucleotides at a 3′-end of the oligonucleotide are modified nucleotides.

14. The method of claim 12, wherein the first to n^{th} nucleotides at the 5′-end of the oligonucleotide or the first to m^{th} nucleotides at the 3′-end of the oligonucleotide are the modified nucleotides, wherein n and m are each independently an integer of 1 to 10.

15. The method of claim 13, wherein the first to n^{th} nucleotides at the 5′-end of the oligonucleotide and the first to m^{th} nucleotides at the 3′-end of the oligonucleotide are the modified nucleotides.

16. The method of claim 15, wherein the oligonucleotide further comprises one or more modified nucleotides between the (n+1)^{th} nucleotide at the 5′-end and the (m+1)^{th} nucleotide at the 3′-end of the oligonucleotide.
